SEQUENCE LISTING (12 sequences)

SEQ ID NO: 1 - protein, 4 aa, synthetic construct
FRRG

SEQ ID NO: 2 - protein, 5 aa, synthetic construct
FRRLG

SEQ ID NO: 3 - protein, 5 aa, synthetic construct
FLRRG

SEQ ID NO: 4 - protein, 6 aa, synthetic construct
FLRRLG

SEQ ID NO: 5 - protein, 6 aa, synthetic construct
FLRRGG

SEQ ID NO: 6 - protein, 6 aa, synthetic construct
FGRRLG

SEQ ID NO: 7 - protein, 6 aa, synthetic construct
FGRRGG

SEQ ID NO: 8 - protein, 5 aa, synthetic construct
FLRRL

SEQ ID NO: 9 - protein, 5 aa, synthetic construct
FGRRL

SEQ ID NO: 10 - protein, 5 aa, synthetic construct
FGRRG

SEQ ID NO: 11 - protein, 4 aa, synthetic construct
FLRR

SEQ ID NO: 12 - protein, 4 aa, synthetic construct
FGRR

(19) United States
(12) Patent Application Publication
(10) Pub. No.: US 2025/0281627 A1
(43) Pub. Date: Sep. 11, 2025

(54) CANCER-TARGETING PEPTIDE, PRODRUG NANOPARTICLES COMPRISING SAME, AND PHARMACEUTICAL COMPOSITION COMPRISING SAME FOR CANCER PREVENTION OR TREATMENT

(71) Applicant: NOXPHARM CO., LTD., Seoul (KR)

(72) Inventors: Kwangmeyung KIM, Seoul (KR); Man Kyu SHIM, Seoul (KR); Hong Yeol YOON, Seoul (KR); Nayeon SHIM, Seoul (KR)

(73) Assignee: NOXPHARM CO., LTD., Seoul (KR)

(21) Appl. No.: 19/127,273

(22) PCT Filed: Sep. 21, 2023
(86) PCT No.: PCT/KR2023/014328

§ 371 Date: May 5, 2025

(30) Foreign Application Priority Data
Nov. 25, 2022 (KR) 10-2022-0160375

(51) Int. Cl. A61K 47/64; A61K 47/69; A61P 35/00; C07K 5/107; C07K 7/06

(52) U.S. Cl. CPC A61K 47/64; A61K 47/6929; A61P 35/00; C07K 5/1016; C07K 7/06

(57) ABSTRACT

The present disclosure relates to a cancer-targeting peptide that can be cleaved by cathepsin B in cancer cells and is characterized by forming prodrug nanoparticles together with an anticancer agent, wherein the preparation of carrier-free prodrug nanoparticles may provide a new approach to cancer treatment and may significantly improve cancer targeting and the therapeutic efficacy of an anticancer agent.

### CROSS REFERENCE TO RELATED APPLICATIONS

This application is a § 371 national stage entry of International Application No. PCT/KR2023/014328, filed on Sep. 21, 2023, which claims priority to Korean Patent Application No. 10-2022-0160375, filed on Nov. 25, 2022, the entire contents of which are incorporated herein by reference.

### SEQUENCE LISTING

The instant application contains a Sequence Listing which has been submitted electronically in a computer readable Sequence Listing XML format and is hereby incorporated by reference in its entirety. Said computer readable Sequence Listing in XML format was created on Nov. 24, 2022, is named G1035-30601_SequenceListing.xml and is 11,426 bytes in size.

### TECHNICAL FIELD

The present disclosure relates to a cancer-targeting peptide, more particularly to a cancer-targeting peptide that can minimize the side effects of an anticancer agent in vivo while providing superior cancer-targeting ability, prodrug nanoparticles containing the same, and a pharmaceutical composition containing the same for preventing or treating cancer.

### BACKGROUND ART

Cancer has been the number one cause of death in Korea for 37 years, and the mortality is increasing every year. Cancer is the only disease with more than 100 deaths, and accounts for 27.5% of all deaths. Cancer is treated by surgery, anticancer chemotherapy and radiation therapy. These treatments are accompanied by severe side effects such as hair loss, diarrhea, death, etc., as compared to the treatments for other diseases.

Therefore, the development of various chemical therapeutic agents that can kill cancer cells selectively has drawn a lot of attention, and targeted therapies targeting oncogenes generated by mutations, etc. have been developed. However, the mutation-targeting cancer therapies could not differentiate cancer from normal tissue 100% due to heterogeneity caused by the somatic mutation of cancer. In addition, the cancer treatment was not so effective because cancer tissues with cancer-specific mutations are only 5-20% of the whole cancer tissues.

In order to solve this problem, therapeutic agents for cancer utilizing the general metabolic specificity of cancer have been studied. They are therapeutic agents for cancer, which treat cancer by finding cancer metabolism and selectively killing cancer cells even under numerous mutations, and thus can be applied to all cancer regardless of the mutations. As representative examples, anticancer agents targeting lactate transporter (MCT1), glucose transporter (glut1), glutaminase 1 (GLS1), acetyl-coA carboxylase (ACC), etc. have been developed. However, the mechanism of cancer metabolism has not been elucidated clearly yet, and most depend on a single activation mechanism. Therefore, they can target only few heterogeneous tumors, and may accelerate drug resistance.

Accordingly, the inventors of the present disclosure have made consistent efforts to elucidate the efficacy of a new conjugate capable of providing targeting characteristics for cancer while minimizing the side effects of the existing anticancer agents. As a result, they have completed the present disclosure by identifying that an optimally designed cancer-targeting peptide exhibits a significant effect of increasing the therapeutic efficacy for cancer while minimizing the side effects of an anticancer agent through conjugation with the anticancer agent.

### REFERENCES OF RELATED ART

### Patent Documents

Patent document 1. Japanese Patent Publication No. 10-2019-0039153.

### DISCLOSURE

### Technical Problem

The present disclosure is directed to providing a cancer-targeting peptide that can be cleaved by cathepsin B in cancer cells.

The present disclosure is also directed to providing a conjugate in which a peptide represented by General Formula 1 is conjugated with an anticancer agent.

The present disclosure is also directed to providing a pharmaceutical composition for preventing or treating cancer, which contains the conjugate as an active ingredient.

### Technical Solution

The present disclosure provides a cancer-targeting peptide represented by General Formula 1, which can be cleaved by cathepsin B in cancer cells.

Phe-[Xaa1]ₙ-Arg-Arg-[Xaa2]ₘ-[Gly]ₒ [General Formula 1]

In the formula 1,
each of Xaa1 and Xaa2 is independently Leu or Gly, and
each of n to o is independently 0 or 1,
with the proviso that n, m and o are not 0 at the same time, and
if n is 0, m is 1 and Xaa2 is Leu.

In the formula 1, n+m+o is 2 or smaller.

In the formula 1, if n is 1, any one of m or o is necessarily 1.

The peptide may be any one of amino acid sequences represented by SEQ ID NOS: 1 to 3.

The peptide may be represented by SEQ ID NO: 1.

The present disclosure also provides a conjugate in which a peptide represented by General Formula 1 is conjugated with an anticancer agent.

Phe-[Xaa1]ₙ-Arg-Arg-[Xaa2]ₘ-[Gly]ₒ [General Formula 1]

In the formula 1,
each of Xaa1 and Xaa2 is independently Leu or Gly, and
each of n to o is independently 0 or 1,
with the proviso that n, m and o are not 0 at the same time, and
if n is 0, m is 1 and Xaa2 is Leu.

In the formula 1, n+m+o is 2 or smaller.

In the formula 1, if n is 1, any one of m or o is necessarily 1.

The peptide may be any one of amino acid sequences represented by SEQ ID NOS: 1 to 3.

The peptide may be represented by SEQ ID NO: 1.

The conjugate may be prepared into a prodrug nanoparticle in a solution through self-assembly.

The prodrug nanoparticle may have an average diameter of 130 to 170 nm.

The proportion of the hydrophobic surface in the entire molecular surface area of the prodrug nanoparticles may be 55 to 60%.

The anticancer agent may be any one selected from a group consisting of Taxol, bendamustine, busulfan, carmustine, chlorambucil, cyclophosphamide, dacarbazine, Adriamycin, daunomycin, ifosfamide, melphalan, procarbazine, streptozocin, temozolomide, asparaginase, capecitabine, cytarabine, 5-fluorouracil, fludarabine, gemcitabine, methotrexate, pemetrexed, raltitrexed, actinomycin D, bleomycin, daunorubicin, doxorubicin, PEGylated liposomal doxorubicin, epirubicin, idarubicin, mitomycin, mitoxantrone, etoposide, docetaxel, irinotecan, paclitaxel, topotecan, vinblastine, vincristine, vinorelbine, carboplatin, cisplatin, oxaliplatin, alemtuzumab, BCG, bevacizumab, cetuximab, denosumab, erlotinib, gefitinib, imatinib, interferon, ipilimumab, lapatinib, panitumumab, rituximab, sunitinib, sorafenib, temsirolimus, trastuzumab, clodronate, ibandronic acid, pamidronate and zoledronic acid

The present disclosure also provides a pharmaceutical composition for preventing or treating cancer, which contains the conjugate as an active ingredient.

The conjugate may be activated as it is cleaved by cathepsin B existing in cancer cells.

The pharmaceutical composition may be accumulated in cancer tissue 2 times or more than in kidney tissue or liver tissue.

The liver may be one or more selected from a group consisting of lung cancer, stomach cancer, glioma, liver cancer, melanoma, kidney cancer, urothelial cancer, head and neck cancer, Merkel cell carcinoma, prostate cancer, blood cancer, breast cancer, mammary gland cancer, colorectal cancer, colon cancer, rectal cancer, pancreatic cancer, brain cancer, ovarian cancer, bladder cancer, bronchial cancer, skin cancer, cervical cancer, endometrial cancer, esophageal cancer, adenocarcinoma of the nasopharynx, thyroid cancer, bone cancer and combinations thereof.

### Advantageous Effects

A cancer-targeting peptide of the present disclosure may provide a new approach to cancer treatment through preparation of carrier-free prodrug nanoparticles by conjugation with an anticancer agent, and may significantly improve cancer targeting and the therapeutic efficacy of the anticancer agent.

In addition, since the prodrug nanoparticles containing the cancer-targeting peptide of the present disclosure exist as nanoparticles in liquid through self-assembly, they can be synthesized and prepared easily and are advantageous for mass production due to high yield and purity.

In addition, the prodrug nanoparticles containing the cancer-targeting peptide of the present disclosure maintain structural stability even after long-term storage, exhibit cancer-specific accumulation by 2 to 16 times or higher in vivo as compared to the existing prodrugs, and have superior biocompatibility with less accumulation in other normal tissues.

In addition, since the prodrug nanoparticles containing the cancer-targeting peptide of the present disclosure are accumulated more in cancer tissue in vivo than in normal tissues even when they are administered repeatedly at high doses, and exist in inactive state even when they are accumulated excessively in normal tissues, they do not cause side effects in vivo and are very suitable for a composition for preventing or treating cancer.

### BRIEF DESCRIPTION OF DRAWINGS

FIG. 1 schematically shows a cancer-targeting peptide according to the present disclosure, a conjugate containing the same, and the structure and in-vivo behavior of prodrug nanoparticles formed from self-assembly of the conjugate.

FIGS. 2A and 2B schematically show the structure of a cancer-targeting peptide according to the present disclosure, and a conjugate containing the same.

FIGS. 3A-3E show the RP-HPLC chromatograms of conjugates prepared in Examples 4 to 6 and Comparative Example 4 (b to e) and a conjugate prepared in Comparative Example 3 (a).

FIG. 4 shows the MALDI-TOF analysis result of conjugates prepared in Examples 4 to 6, Comparative Example 3 and Comparative Example 4. CHCA (cyano-4-hydroxycinnamic acid) was used for the analysis (molecular weight range=500 to 2,000 Da).

FIG. 5 shows the DLS analysis result for prodrug nanoparticles of Examples 4 to 6 and prodrug nanoparticles of Examples 3 and 4.

FIG. 6 shows the size and size distribution of prodrug nanoparticles of Examples 4 to 6 and prodrug nanoparticles of Examples 3 and 4 in saline.

FIG. 7 shows the result of evaluating the colloidal stability of prodrug nanoparticles prepared in Examples 4 to 6 and prodrug nanoparticles prepared in Comparative Examples 3 to 4 in saline (mean±SD).

FIG. 8 shows the result of measuring the proportion of hydrophobic surface for prodrug nanoparticles of Examples 4 to 6 and prodrug nanoparticles of Comparative Examples 3 to 4.

FIG. 9 shows the intermolecular dynamic simulation result for prodrug nanoparticles of Examples 4 to 6 and prodrug nanoparticles of Comparative Examples 3 to 4.

FIG. 10 shows the TEM images of prodrug nanoparticles of Examples 4 to 6 and prodrug nanoparticles of Comparative Example 3.

FIG. 11 shows the RP-HPLC chromatograms for prodrug nanoparticles of Examples 4 to 6 and prodrug nanoparticles of Comparative Example 3 in a 1 mM cathepsin B solution depending on time.

FIG. 12 shows the result of calculating peptide degradation ratio (%) by quantifying the RP-HPLC chromatograms in FIG. 11 depending on time.

FIG. 13A shows the confocal microscopic images for drug administration groups 1, 2 and 3 (FRRL-DOX, FRRLG-DOX, FLRRG-DOX) and a control group (FRRG-DOX) obtained after treating HT29 cells with prodrug nanoparticles of Examples 4 to 6 and prodrug nanoparticles of Comparative Example 3.

FIG. 13B shows the result of comparing the fluorescence intensity of the anticancer agent (DOX) in the nucleus and cytoplasm from the confocal fluorescence images (48 h) of the drug administration groups 1, 2 and 3 (FRRL-DOX, FRRLG-DOX, FLRRG-DOX) and the control group (FRRG-DOX) obtained after treating HT29 cells with the prodrug nanoparticles of Examples 4 to 6 and the prodrug nanoparticles of Comparative Example 3 (mean±SD, n=3).

FIG. 13C shows the result of analyzing the anticancer agent (DOX) from filtrates of the drug administration groups 1, 2 and 3 (FRRL-DOX, FRRLG-DOX, FLRRG-DOX) and the control group (FRRG-DOX) obtained after treating HT29 cells with the prodrug nanoparticles of Examples 4 to 6 and the prodrug nanoparticles of Comparative Example 3 by MALDI-TOF.

FIG. 14A shows the result of analyzing the expression level of cathepsin B in HT29 cells and H9C2 cells by western blot (top) and the result of quantifying band intensities (bottom).

FIG. 14B shows the confocal microscopic images of a single administration group (DOX), a drug administration group (Example 4; FRRL-DOX) and a control group (Comparative Example 3; FRRG-DOX) for H9C2 cells and HT29-Inh cells.

FIG. 14C shows the result of quantifying the fluorescence intensity of the anticancer agent (DOX) measured from the images of FIG. 14b.

FIG. 14D shows the result of measuring the cell survivability of HT29 cells for the single administration group a (DOX), the drug administration group (Example 4; FRRL-DOX) and the control group (Comparative Example 3; FRRG-DOX) depending on concentration.

FIG. 14E shows the result of measuring the cell survivability of H9C2 cells for the single administration group (DOX), the drug administration group (Example 4; FRRL-DOX) and the control group (Comparative Example 3; FRRG-DOX) depending on concentration.

FIG. 15A shows the digital fluorescence microscopic images for test groups 1, 2 and 4 obtained 9 hours after injection of an anticancer agent (DOX) (4 mg/kg) or prodrug nanoparticles of Example 4 or Comparative Example 3 (4 mg/kg based on DOX concentration) to an animal model of cancer. FIG. 15B shows the fluorescence intensity of cancer tissue quantitated from the images of FIG. 15A.

FIG. 15C shows the result of isolating cancer tissue from the test groups 1, 2 and 4 and analyzing fluorescence intensity.

FIG. 15D shows the fluorescence microscopic images of the tissues of major organs (liver, lung, spleen, kidney, heart and tumor) from the test group 1 to 5.

FIG. 15E shows the result of isolating the tissues of major organs (liver, lung, spleen, kidney, heart and tumor) from the test group 1 to 5 and analyzing fluorescence intensity.

FIG. 15F shows the confocal microscopic images of cancer tissues isolated from the test groups 1, 2 and 4 obtained after staining with DAPI.

FIG. 15G shows the result of quantifying the fluorescence intensity of the anticancer agent (DOX) in cancer tissues isolated from the test groups 1, 2 and 4.

FIG. 16A shows the result of measuring the tumor volume for test groups 6 to 8 depending on time.

FIG. 16B shows the result of measuring survival rate for the test groups 6 to 8 depending on time.

FIG. 16C shows the TUNEL assay and histopathological analysis results for cancer tissues isolated from the test groups 6 to 8.

FIG. 17 shows the result of measuring body weight change for a normal group and test groups 9 to 12.

FIG. 18 shows the histochemical analysis result of the liver, lung, spleen, kidney and heart tissues of a normal group and test groups 9 to 12 stained with H&E.

FIG. 19 shows the result of measuring body weight change for a normal group and test groups 13 to 16.

FIG. 20 shows the result of the histochemical analysis result of the liver, lung, spleen, kidney and heart tissues of a normal group and test groups 13 to 16 stained with H&E.

FIG. 21 shows the result of evaluating hemocompatibility for a normal group and test groups 9 to 12.

FIG. 22 shows the result of evaluating hemocompatibility for a normal group and test groups 13 to 16.

### BEST MODE

The present disclosure will now be described in detail.

A prodrug refers to a drug which is different from existing medications in chemical structure or essential composition and exhibits efficacy as it is metabolized within the body. The prodrug is advantageous in that the inactive state can be maintained effectively by inhibiting the undesirable pharmacological action of the drug, but is limited in that bioavailability is low and its efficacy cannot be achieved sufficiently because it is removed quickly from the blood circulation.

Nanoparticles conjugated with various carriers have been studied to extend the circulation time of the prodrug. Although it was expected that the concentration of the drug accumulated in the target tissue (e.g., cancer tissue) would be increased due to the increased circulation time, many problems such as unexpected side effects occurred due to low intracellular delivery efficiency of lower than 5%, drug dose limited to 10% or lower due to the presence of the carrier, and intrinsic toxicity and immunogenicity of the substances constituting the nanoparticles.

Therefore, the inventors of the present disclosure have made consistent efforts to develop a new carrier-free conjugate with a small molecular weight, which exhibits superior cancer-targeting characteristics while minimizing side effects. As a result, they have revealed a new cancer-targeting peptide and completed the present disclosure by identifying that prodrug nanoparticles containing the same can significantly increase the therapeutic efficacy of an anticancer agent for cancer while minimizing side effects.

An aspect of the present disclosure relates to a cancer-targeting peptide represented by General Formula 1, which can be cleaved by cathepsin B in cancer cells.

Phe-[Xaa1]ₙ-Arg-Arg-[Xaa2]ₘ-[Gly]ₒ [General Formula 1]

In the formula 1,
each of Xaa1 and Xaa2 is independently Leu or Gly,
each of n to o is independently 0 or 1,
with the proviso that n, m and o are not 0 at the same time, and
if n is 0, m is 1 and Xaa2 is Leu.

Since the cancer-targeting peptide presented in the present disclosure has superior ability of targeting cancer tissue and cancer cells, it can also be used for diagnosis of cancer.

Through conjugation with a drug, the cancer-targeting peptide presented in the present disclosure can self-assemble into spherical nanoparticles via π-π staking with the drug and hydrophobic interactions due to amphiphilic characteristics, without negatively affecting the pharmacological effect of the drug, significantly inhibit toxicity for normal tissues by stably maintaining the drug in an inactive state, and improve the delivery and accumulation of the drug into cancer cells/cancer tissues.

In the present disclosure, the peptide refers to a linear molecule formed as amino acid residues are bonded with each other via peptide bonding. Representative amino acids and their acronyms are: alanine (Ala, A), isoleucine (Ile, I), leucine (Leu, L), methionine (Met, M), phenylalanine (Phe, F), proline (Pro, P), tryptophan (Trp, W), valine (Val, V), asparagine (Asn, N), cysteine (Cys, C), glutamine (Gln, Q), glycine (Gly, G), serine (Ser, S), threonine (Thr, T), tyrosine (Try, Y), aspartic acid (Asp, D), glutamic acid (Glu, E), arginine (Arg, R), histidine (His, H) and lysine (Lys, K).

The peptide represented by General Formula 1 of the present disclosure may be 4 to 5 amino acids long. The Xaa1, Xaa2 and Gly amino acid residues in General Formula 1 include “gaps” that may not be known or labeled. Although the gaps were not specified under the Rule 37 of the World Intellectual Property Organization (WIPO) Standard ST.26, the sequence represented by General Formula 1 may any amino acid sequence selected from a group consisting of SEQ ID NOS: 1 to 12.

That is, the peptide represented by General Formula 1 may have any amino acid sequence selected from a group consisting of SEQ ID NOS: 1 to 12.

Specifically, in a specific exemplary embodiment of the present disclosure, in General Formula 1 of the present disclosure, n+m+o may be 2 or smaller. For example, the peptide may be represented by any amino acid sequence selected from a group consisting of SEQ ID NOS: 1 to 3 and 8 to 12.

Specifically, in a specific exemplary embodiment of the present disclosure, General Formula 1 of the present disclosure, if n is 1, any one of m or o is necessarily 1. For example, the peptide may be represented by any amino acid sequence selected from a group consisting of SEQ ID NOS: 1 to 3 and 8 to 10.

When designing the cancer-targeting peptide according to the present disclosure, it is important that the peptide forms a conjugate with an anticancer agent as it is specifically cleaved by the enzyme cathepsin B, and self-assembles into spherical nanoparticles in a solution. For this, the cancer-targeting peptide of the present disclosure may be specifically any one of amino acid sequences represented by SEQ ID NOS: 1 to 3.

Most specifically, the cancer-targeting peptide may be a peptide represented by SEQ ID NO: 1. In this case, cancer-targeting ability may be superior and the effect of reducing side effects for normal tissue tissues in vivo through conjugation with an anticancer agent may be the most superior, as described later in the test examples.

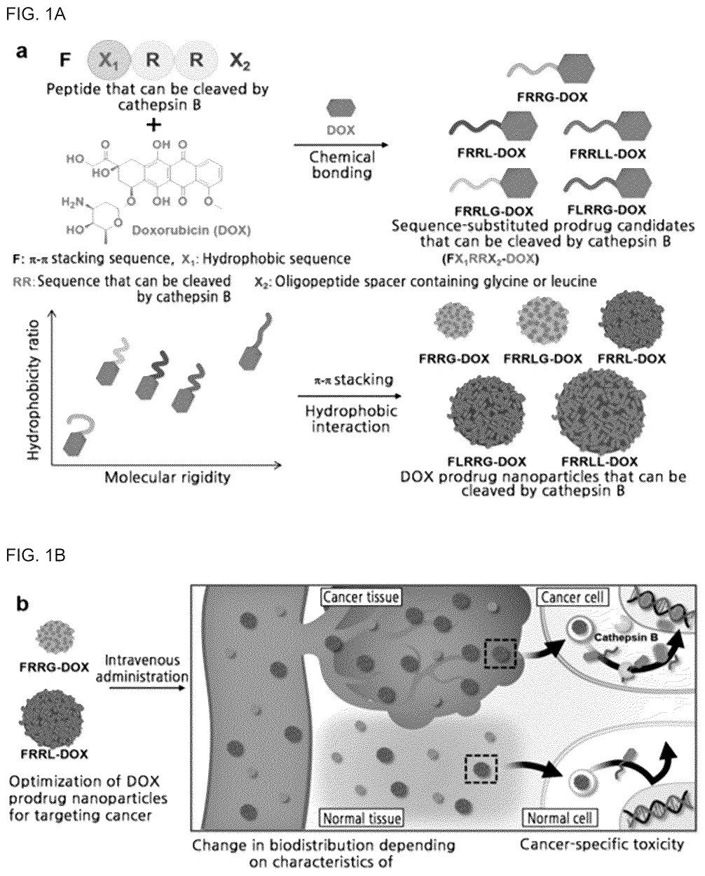

Another aspect of the present disclosure relates to a conjugate in which a peptide represented by General Formula 1 is conjugated with an anticancer agent. The specific structure and the mechanism of action in cancer tissues/cancer cells in vivo are schematically shown in FIG. 1.

Phe-[Xaa1]ₙ-Arg-Arg-[Xaa2]ₘ-[Gly]ₒ [General Formula 1]

In the formula 1,
each of Xaa1 and Xaa2 is independently Leu or Gly,
each of n to o is independently 0 or 1,
with the proviso that n, m and o are not 0 at the same time, and
if n is 0, m is 1 and Xaa2 is Leu.

Although the existing anticancer drug conjugates contain a linker between a peptide and an anticancer agent to provide flexibility between the anticancer agent and the peptide, the conjugate according to the present disclosure can provide sufficient flexibility with the anticancer agent without containing a linker due to the peptide represented by General Formula 1. Therefore, it is very favorable for mass production since the synthesis procedure is simple. In addition, the conjugate exhibits high purity and yield despite a simple structure, and has superior structural stability in solution and superior pharmacological effect in vivo.

The peptide represented by General Formula 1 of the present disclosure may be 4 to 5 amino acids long. It may be represented by any amino acid sequence selected from a group consisting of SEQ ID NOS: 1 to 12.

In a specific exemplary embodiment of the present disclosure, in General Formula 1 of the present disclosure, n+m+o is specifically 2 or smaller. For example, the peptide may be represented by any amino acid sequence selected from a group consisting of SEQ ID NOS: 1 to 3 and 8 to 12.

Specifically, in a specific exemplary embodiment of the present disclosure, in General Formula 1 of the present disclosure, if n is 1, any of m or o is necessary 1. For example, the peptide may be represented by any amino acid sequence selected from a group consisting of SEQ ID NOS: 1 to 3 and 8 to 10.

When designing a conjugate of the cancer-targeting peptide according to the present disclosure and an anticancer agent, it is important that the peptide self-assembles into spherical nanoparticles in a solution as it is specifically cleaved by the enzyme cathepsin B. For this, the cancer-targeting peptide of the present disclosure is more specifically any one of amino acid sequences represented by SEQ ID NOS: 1 to 3.

In the conjugate according to the present disclosure, the peptide is most specifically a peptide represented by SEQ ID NO: 1. In this case, cancer-targeting ability may be superior and the effect of reducing side effects for normal tissue tissues in vivo through conjugation with an anticancer agent may be the most superior, as described later in the test examples.

The conjugate self-assembles into prodrug nanoparticles in a solution. The nanoparticles have an average diameter of 140 to 160 nm in distilled water, and have an average diameter of 130 to 170 nm in physiological saline.

If the average diameter of the prodrug nanoparticles in physiological saline is smaller than 130 nm, accumulation efficiency in normal tissues other than cancer tissues may increase. And, if it exceeds 170 nm, pharmacological effect may decrease by 2 times or more due to insufficient cellular uptake.

For both the conjugate of the present disclosure and the prodrug nanoparticles formed therefrom, the anticancer agent is separated and activated as the peptide is cleaved in the presence of the cathepsin B enzyme. The separated anticancer agent is completely degraded by lysosomes present in cancer cells, and is absorbed and delivered into the nuclei of the cancer cells to induce the death of the cancer cells. The cleaved peptide fragment, which is nontoxic and stable in itself, is completely degraded in vivo and participates in metabolic processes in the body or is released out of the body through the kidneys.

Since cathepsin B is hardly secreted in non-cancer cells but is expressed specifically in cancer cells, the conjugate of the present disclosure targeting the same remains in inactive state in non-cancer cells and does not cause toxicity in normal cells.

In addition, the conjugate of the present disclosure and the prodrug nanoparticles formed therefrom do not respond to enzymes or proteases other than cathepsin B. In addition, since the conjugate of the present disclosure and the prodrug nanoparticles formed therefrom target microenvironments, not oncogenes, they can be applied not just to specific cancers, but broadly to resistant cancers, metastatic cancers, mutated cancers, etc.

The proportion of the hydrophobic surface in the entire molecular surface area of the prodrug nanoparticles may be specifically smaller than 60%, most specifically 55% to 60%. If the proportion of the hydrophobic surface exceeds 60%, the structure of the spherical nanoparticles may not be maintained under physiological conditions (e.g., saline), and pharmacological effect may decrease significantly as the average diameter of the particles exceeds 1000 nm. And, if it is smaller than 55%, dispersion stability may decrease under physiological conditions (e.g., saline), and cancer-targeting efficiency may decrease due to decreased circulation time.

The proportion of the hydrophobic surface may be analyzed with the Desmond module of the Schrödinger suite using the following parameters: OPLS4; solvent model: TIP5P; ion placement: chloride; boundary conditions: orthorhombic box shape, box size calculation method (buffer); simulation time: 100 ns; approximate number of frames: 100; ensemble class: NPT; temperature: 300K; pressure: 1.01325 bar; thermostat method: Nose-Hoover chain; coulombic interaction cutoff radius: 9.0 Å.

The anticancer agent is not specially limited as long as it is a molecule having the effect of preventing, treating or killing cancer used in the art. For example, it may be any one selected from a group consisting of Taxol, bendamustine, busulfan, carmustine, chlorambucil, cyclophosphamide, dacarbazine, Adriamycin, daunomycin, ifosfamide, melphalan, procarbazine, streptozocin, temozolomide, asparaginase, capecitabine, cytarabine, 5-fluorouracil, fludarabine, gemcitabine, methotrexate, pemetrexed, raltitrexed, actinomycin D, bleomycin, daunorubicin, doxorubicin, PEGylated liposomal doxorubicin, epirubicin, idarubicin, mitomycin, mitoxantrone, etoposide, docetaxel, irinotecan, paclitaxel, topotecan, vinblastine, vincristine, vinorelbine, carboplatin, cisplatin, oxaliplatin, alemtuzumab, BCG, bevacizumab, cetuximab, denosumab, erlotinib, gefitinib, imatinib, interferon, ipilimumab, lapatinib, panitumumab, rituximab, sunitinib, sorafenib, temsirolimus, trastuzumab, clodronate, ibandronic acid, pamidronate and zoledronic acid, specifically doxorubicin.

Another aspect of the present disclosure relates to a pharmaceutical composition for preventing or treating cancer, which contains the conjugate as an active ingredient.

In the present disclosure, ‘cancer or tumor’ specifically refers to a group of diseases wherein lumps or tumors are formed of undifferentiated cells that proliferate unlimited in tissues, infiltrate into and destroy nearby normal tissues or organs, metastasize from primary sites to other organs, and take out individuals' lives.

The cancer may be solid cancer or blood cancer, primary cancer or metastatic cancer, or mutated cancer caused by mutation of specific genes. The mutated cancer may be KRAS-mutated cancer.

The cancer may be one or more selected from a group consisting of brain cancer, lung cancer, stomach cancer, glioma, liver cancer, melanoma, head and neck cancer, Merkel cell carcinoma, blood cancer, breast cancer, mammary gland cancer, colorectal cancer, colon cancer, rectal cancer, pancreatic cancer, anal cancer, ovarian cancer, bladder cancer, bronchial cancer, skin cancer, cervical cancer, endometrial cancer, esophageal cancer, nasopharyngeal cancer, thyroid cancer, bone cancer, gallbladder cancer, lymphoma, osteosarcoma, oral cancer, bronchial cancer, laryngeal cancer, skin cancer, squamous cell carcinoma, parathyroid cancer, ureter cancer, kidney cancer, prostate cancer and urothelial cancer, although not being limited thereto.

The pharmaceutical composition according to the present disclosure may further contain a suitable carrier, excipient and diluent commonly used for preparation of pharmaceutical compositions. The excipient may be, for example, one or more selected from a group consisting of a diluent, a binder, a disintegrant, a glidant, an absorbent, a moisturizer, a film-coating substance, and a controlled-release additive.

The pharmaceutical composition according to the present disclosure may be in the form of a powder, a granule, a controlled-release granule, an enteric-coated granule, a liquid, a collyrium, an elixir, an emulsion, a suspension, a spirit, a troche, an aromatic water, a lemonade, a tablet, a controlled-release tablet, an enteric-coated tablet, a sublingual tablet, a hard capsule, a soft capsule, a controlled-release capsule, an enteric-coated capsule, a pill, a tincture, a soft extract, a dry extract, a fluid extract, an injection, a capsule, a perfusion, a plaster, a lotion, a paste, a spray, an inhalant, a patch, a sterile injection solution, an aerosol, a formulation for external application, etc. according to common methods. The formulation for external application may be a cream, a gel, a patch, a spray, an ointment, a plaster, a lotion, a liniment, a paste, a cataplasm, etc.

The carrier, excipient and diluent that may be contained in the pharmaceutical composition according to the present disclosure may be lactose, dextrose, sucrose, oligosaccharide, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate or mineral oil.

For formulation, a commonly used diluent or excipient such as a filler, an extender, a binder, a wetting agent, a disintegrant, a surfactant, etc. is used.

In a tablet, powder, granule, capsule, pill or troche according to the present disclosure, an excipient such as corn starch, potato starch, wheat starch, lactose, white sugar, glucose, fructose, D-mannitol, precipitated calcium carbonate, synthetic aluminum silicate, calcium monohydrogen phosphate, calcium sulfate, sodium chloride, sodium bicarbonate, purified lanolin, microcrystalline cellulose, dextrin, sodium alginate, methyl cellulose, sodium carboxymethyl cellulose, kaolin, urea, colloidal silica gel, hydroxypropyl starch, hydroxypropyl methyl cellulose, 1928, 2208, 2906, 2910, propylene glycol, casein, calcium lactate, sodium starch glycolate, etc.; a binder such as gelatin, gum arabic, ethanol, agar powder, cellulose acetate phthalate, carboxymethyl cellulose, carboxymethyl cellulosecalcium, glucose, purified water, sodium caseinate, glycerin, stearic acid, sodium carboxymethyl cellulose, sodium methyl cellulose, methyl cellulose, microcrystalline cellulose, dextrin, hydroxyl cellulose, hydroxypropyl starch, hydroxymethyl cellulose, purified shellac, starch paste, hydroxypropyl cellulose, hydroxypropyl methyl cellulose, polyvinyl alcohol, polyvinylpyrrolidone, etc., a disintegrant such as hydroxypropyl methyl cellulose, corn starch, agar powder, methyl cellulose, bentonite, hydroxypropyl starch, sodium carboxymethyl cellulose, sodium alginate, carboxymethyl cellulosecalcium, calcium citrate, sodium lauryl sulfate, anhydrous silicic acid, 1-hydroxypropyl cellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelated starch, gum arabic, amylopectin, pectin, sodium polyphosphate, ethyl cellulose, white sugar, magnesium aluminum silicate, D-sorbitol, crystalline anhydrous silicic acid, etc.; or a glidant such as calcium stearate, magnesium stearate, stearic acid, hydrogenated vegetable oil, talc, lycopodium, kaolin, vaseline, sodium stearate, cocoa butter, sodium salicylate, magnesium salicylate, polyethylene glycol 4000 and 6000, liquid paraffin, hydrogenated soybean oil (Lubri wax), aluminum stearate, zinc stearate, sodium lauryl sulfate, magnesium oxide, macrogol, synthetic aluminum silicate, anhydrous silicic acid, higher fatty acid, higher alcohol, silicone oil, paraffin oil, polyethylene glycol fatty acid ether, starch, sodium chloride, sodium acetate, sodium oleate, D/L-leucine, crystalline anhydrous silicic acid, etc. may be used as an additive.

In a liquid according to the present disclosure, water, dilute hydrochloric acid, dilute sulfuric acid, sodium citrate, sucrose monostearate, polyoxyethylene sorbitol fatty acid ester (Tween ester), polyoxyethylene monoalkyl ether, lanolin ether, lanolin ester, acetic acid, hydrochloric acid, ammonia water, ammonium carbonate, potassium hydroxide, sodium hydroxide, prolamin, polyvinylpyrrolidone, ethyl cellulose, sodium carboxymethyl cellulose, etc. may be used as an additive.

In a syrup according to the present disclosure, a solution of white sugar, other sugars, a sweetener, etc. may be used and, if necessary, an aromatic, a colorant, a preservative, a stabilizer, a suspending agent, an emulsifier, a thickener, etc. may be used.

In an emulsion according to the present disclosure, purified water may be used and, if necessary, an emulsifier, a preservative, a stabilizer, an aromatic, etc. may be used.

In a suspension according to the present disclosure, a suspending agent such as acacia, tragacanth, methyl cellulose, carboxymethyl cellulose, sodium carboxymethyl cellulose, microcrystalline cellulose, sodium alginate, hydroxypropyl methyl cellulose, 1828, 2906, 2910, etc. may be used and, if necessary, a surfactant, a preservative, a stabilizer, a colorant or an aromatic may be used.

An injection according to the present disclosure may contain a solvent such as distilled water for injection, 0.9% sodium chloride injection, Ringer's injection, dextrose injection, dextrose+sodium chloride injection, PEG, lactated Ringer's injection, ethanol, propylene glycol, non-volatile sesame oil, cottonseed oil, peanut oil, soybean oil, corn oil, ethyl oleate, isopropyl myristate and methyl benozate; a solubilizing aid such as sodium benzoate, sodium salicylate, sodium acetate, urea, urethane, monoethylacetamide, butazolidine, propylene glycol, Tween, nicotinamide, hexamine and dimethylacetamide; a buffer such as a weak acid and its salt (acetic acid and sodium acetate), a weak base and its salt (ammonia and ammonium acetate), an organic compound, a protein, albumin, peptone and gum; an isotonic agent such as sodium chloride; a stabilizer such as sodium bisulfite (NaHSO₃), carbon dioxide gas, sodium thiosulfate (Na₂S₂O₃), sodium sulfite (Na₂SO₃), nitrogen gas (N₂) and ethylenediaminetetraacetic acid; an antioxidant such as 0.1% sodium bisulfide, sodium formaldehyde sulfoxylate, thiourea, disodium ethylenediaminetetraacetate and acetone sodium bisulfite; an analgesic such as benzyl alcohol, chlorobutanol, procaine hydrochloride, glucose and calcium gluconate; or a suspending agent such as sodium CMC, sodium alginate, Tween80 and monoaluminum stearate.

In a suppository according to the present disclosure, a base such as cocoa butter, lanolin, witepsol, polyethylene glycol, glycerogelatin, methyl cellulose, carboxymethyl cellulose, a mixture of stearic acid and oleic acid, subanal, cottonseed oil, peanut oil, palm oil, cocoa butter+cholesterol, lecithin, Lanette wax, glycerol monostearate, Tween or Span, Imhausen, monolene (propylene glycol monostearate), glycerin, adeps solidus, Butyrum Tego-G, Cebes Pharma 16, Hexaride base 95, Cotomar, Hydrokote SP, S-70-XXA, S-70-XX75(S-70-XX95), Hydrokote(Hydrokote) 25, Hydrokote 711, Idropostal, Massa estrarium, A, AS, B, C, D, E, I and T, Massa-MF, Marsupol, Marsupol-15, Neosupostal-N, Paramound-B, Suposiro (OSI, OSIX, A, B, C, D, H and L), suppository base type IV (AB, B, A, BC, BBG, E, BGF, C, D, 299), Supostal (N, Es), Wecobi (W, R, S, M, Fs), and Tegester triglyceride base (TG-95, MA, 57) may be used.

Solid formulations for oral administration include a tablet, a pill, a powder, a granule, a capsule, etc. They are prepared by mixing the active ingredient with at least one excipient, e.g., starch, calcium carbonate, sucrose, lactose, gelatin, etc. In addition to the simple excipients, a lubricant such as magnesium stearate and talc is also used.

Liquid formulations for oral administration include a suspension, an internal solution, an emulsion, a syrup. In addition to a commonly used simple diluent such as water or liquid paraffin, various excipients, e.g., a wetting agent, a sweetener, an aromatic, a preservative, etc. may be contained. Formulations for parenteral administration include a sterilized aqueous solution, a nonaqueous solution, a suspension, an emulsion, a freeze-dried formulation and a suppository. As the nonaqueous solution or suspension, propylene glycol, polyethylene glycol, vegetable oil such as olive oil, injectable ester such as ethyl oleate, etc. may be used.

The pharmaceutical composition according to the present disclosure is administered in a pharmaceutically effective amount. In the present disclosure, the “pharmaceutically effective amount” refers to an amount sufficient to treat a disease with a reasonable benefit/risk ratio applicable to medical treatment, and the level of the effective amount may be determined according to the type of a patient's disease, the severity of the disease, the activity of a drug, the sensitivity to the drug, the time of administration, the route of administration, excretion rate, the duration of treatment, factors including drugs used concurrently, and other factors well known in the medical field.

The content of the active ingredient in the pharmaceutical composition may be adjusted appropriately depending on the purpose of use of the pharmaceutical composition, the type of formulation, etc. For example, it may be 0.001 to 99 wt %, 0.001 to 90 wt %, 0.001 to 50 wt %, 0.01 to 50 wt %, 0.1 to 50 wt %, or 1 to 50 wt %, based on the total weight of the pharmaceutical composition, although not being limited thereto.

The administration dosage of the pharmaceutical composition of the present disclosure may vary variously depending on many factors including the activity of the active ingredient, the age, body weight, general health conditions, sex and diet of the patient, the time of administration, the route of administration, excretion rate, combination with other drugs, and the severity of the specific disease to be prevented or treated. The administration dosage of the pharmaceutical composition may be selected adequately by those skilled in the art although it varies depending on the patient's condition and body weight, the severity of the disease, the type of the drug, the route of administration, and the duration of treatment. For example, a daily administration dosage may be 0.0001 mg/kg to 100 mg/kg, specifically 0.001 mg/kg to 100 mg/kg, more specifically 0.01 mg/kg to 100 mg/kg, furthermore specifically 0.1 mg/kg to 100 mg/kg, even more specifically 1 mg/kg to 100 mg/kg, most specifically 5 mg/kg to 100 mg/kg. The administration may be made once or several times a day. The above-described administration dosage does not limit the scope of the present disclosure in any way. The pharmaceutical composition of the present disclosure is advantageous in that it exhibits few side effects even after repeated administration, unlike the existing anticancer agents.

The pharmaceutical composition according to the present disclosure may be administered as an individual therapeutic agent or in combination with other therapeutic agents. It may be administered sequentially or concurrently with a conventional therapeutic agent, and may be administered once or multiple times. It is important to administer the pharmaceutical composition in an amount sufficient to obtain the maximum effect with the minimum amount without side effects in consideration of all the above-mentioned factors, which may be easily determined by a person having ordinary skill in the technical field to which the present disclosure belongs.

The pharmaceutical composition of the present disclosure may be administered to a subject through various routes. All modes of administration may be expected and may include, for example, oral administration, subcutaneous injection, intraperitoneal administration, intravenous injection, intramuscular injection, perispinal (intradural) injection, sublingual administration, buccal administration, rectal insertion, vaginal insertion, intraocular administration, auricular administration, intranasal administration, inhalation, spraying through the mouth or nose, intradermal administration, transdermal administration, etc. Specifically, it may be administered by subcutaneous injection, intraperitoneal administration, intramuscular injection, perispinal (intradural) injection or intravenous injection.

The pharmaceutical composition of the present disclosure may be determined depending on the type of the drug as an active ingredient together with various related factors such as the disease to be treated, the rout of administration, the age, sex and body weight of the patient, the severity of the disease, etc.

In the present disclosure, the ‘subject’ refers to a subject in need of treatment of a disease, more specifically a mammal such as a human or non-human primate, mouse, rat, dog, cat, horse, cow, etc., although not being limited thereto.

In the present disclosure, ‘administration’ refers to any action of providing the composition of the present disclosure to a subject using any suitable method.

In the present disclosure, ‘prevention’ refers to any action of suppressing or delaying the onset of a disease, ‘treatment’ refers to any action of improving or beneficially changing a disease and metabolic abnormalities resulting therefrom by administrating the pharmaceutical composition according to the present disclosure, and ‘alleviation’ refers to any action of reducing parameters associated with a disease, e.g., the severity of a symptom, by administrating the pharmaceutical composition according to the present disclosure.

Hereinafter, the present disclosure will be described in more detail through specific examples. However, the examples are provided only to describe the present disclosure more specifically, and it will be obvious to those having ordinary knowledge in the art that the scope of the present disclosure is not limited by them.

### Experimental Materials

Doxorubicin hydrochloride (DOX; >99%), 1-ethyl-3-(3-dimethylaminopropyl)carbodiimide hydrochloride (EDC; 98%), N-hydroxysuccinimide (NHS; 98%), N,N-diisopropylethylamine (DIPEA; ≥99%), N,N-dimethylformamide (DMF; anhydrous, 99.8%), and dimethyl sulfoxide (DMSO; anhydrous, ≥99.9%) were purchased from Sigma-Aldrich (St. Louis, MO, USA).

Five peptides represented by SEQ ID NOS: 1 to 5 were synthesized by Peptron Co. (Daejeon, Korea). The peptides were acylated at the N-terminus: SEQ ID NO: 1; Phe-Arg-Arg-Leu (FRRL), SEQ ID NO: 2; Phe-Arg-Arg-Leu-Gly (FRRLG), SEQ ID NO: 3; Phe-Leu-Arg-Arg-Gly (FLRRG), SEQ ID NO: 13; Phe-Arg-Arg-Gly (FRRG) and SEQ ID NO: 14; Phe-Arg-Arg-Leu-Leu (FRRLL).

Cathepsin B was purchased from R&D Systems (Minneapolis, MN, USA), and DMEM (Dulbecco's modified Eagle's medium), RPMI 1640 medium, FBS (fetal bovine serum), penicillin and streptomycin were purchased from WELGENE Inc. (Daegu, Korea).

Cathepsin B monoclonal antibody and Z-Phe-Ala-FMK (benzyloxycarbonyl-Phe-Ala-fluoromethylketone) were purchased from Santa Cruz Biotechnology, Inc. (Dallas, TX, USA), and a RIPA (radio-immunoprecipitation assay) buffer, a BCA (bicinchoninic acid) protein quantification kit and streptavidin-HRP (horseradish peroxidase) were purchased from Thermo Fisher Scientific Inc. (Rockford, IL, USA). 5-week-old male BALB/c and BALB/c nu/nu mice were purchased from NaraBio, Inc. (Seoul, Korea). All chemicals were used without further purification.

HT29 (human colon adenocarcinoma) cells and H9C2 (rat BDIX heart myoblast) cells used in the present disclosure were acquired from the American Type Culture Collection (ATCC; Rockville, MD, USA), and were cultured in RPMI-1640 containing 10% (v/v) FBS (fetal bovine serum; GenDEPOT, Barker, TX, USA) or in DMEM containing 1% (v/v) streptomycin and 100 U/mL penicillin.

### Experimental Equipment

The synthesis and degradation of a conjugate or prodrug nanoparticles were confirmed by RP-HPLC (1200 series Agilent Technologies, USA). The molecular mass of the prodrug nanoparticles was measured by MALDI-TOF (Voyager DE-STR, Applied Biosystems, Foster City, CA, USA) using a CHCA matrix. The size, shape and polydispersity of the prodrug nanoparticles were analyzed by DLS (Zetasizer Nano ZS, Malvern Instruments, Malvern, UK) and TEM (Tecnai F20 G2, Field Electron and Ion Company, Hillsboro, OR, USA).

A CLSM (confocal laser scanning microscope; Leica TCS SP8, Leica Microsystems GmbH; Wetzlar, Germany), a flow cytometer (BD FACSVerse, BD Bioscience, San Jose, CA, USA) and a microplate reader (VERSAmax™; Molecular Devices Corp., Sunnyvale, CA, USA) were used for in-vitro experiment. For in-vivo and ex-vivo experiments, an IVIS Lumina Series III system (PerkinElmer, Waltham, MA, USA) equipped with a living Image software (PerkinElmer) was used. The experimental equipment described above may be modified adequately depending on experiments.

### EXAMPLES AND COMPARATIVE EXAMPLES

### Examples 1 to 3. Preparation of Cancer-Targeting Peptide

Three different sequences (FRRL (SEQ ID NO: 1), FRRLG (SEQ ID NO: 2), FLRRG (SEQ ID NO: 3)) were prepared to confirm the most adequate cathepsin B-cleavable peptide sequences in the formation of the prodrug nanoparticles. The peptides were acylated at the N-terminus through Fmoc solid-phase synthesis by Peptron Co.

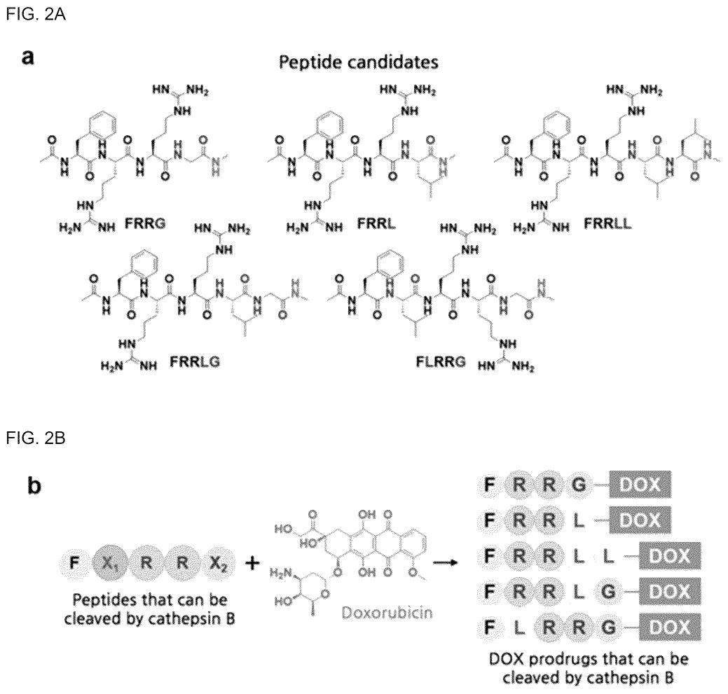

FIG. 2a shows the structure of the cancer-targeting peptides. The N-terminus of each cancer-targeting peptide was protected with an acetate group in order to prevent unwanted reaction during conjugation with an anticancer agent.

Through various attempts on designing the cancer-targeting peptide, it was confirmed that it is advantageous that the peptide includes Phe and Arg-Arg sequences for cathepsin B-specific cleavage and formation of nanoparticles through self-assembly. When the peptide does not include the sequences, it was not cleaved by cathepsin B or did not form nanoparticles in a solution. Through this process, three cancer-targeting peptides including or substituted with a Gly and/or Leu spacer were selected finally.

### Examples 4 to 6. Preparation of Conjugate

A conjugate of the peptide according to the present disclosure and an anticancer agent is prepared by chemically conjugating an anticancer agent at the C-terminus of the peptide through amide bonding using EDC and NHS (see FIG. 2b).

Specifically, EDC (950 mg, 4.96 mmol), NHS (350.0 mg, 3.04 mmol) and DOX (640.0 mg, 1.10 mmol) were added to a 250-mL 2-neck round-bottom flask, and dissolved by adding 100 mL of anhydrous DMF. After dissolving each of the peptides synthesized in Examples 1 to 3 (1.56 mmol) in anhydrous DMF (100 mL), the mixture was added to the above flask. Then, DIPEA (29.6 μL, 0.02 mmol) was added. After 12 hours of reaction at 0° C., a product was precipitated from the mixture in cold diethyl ether, and filtered through a 5-μm semi-preparative C18 column (150 mm×20 mm; YMC, Dinslaken, Germany). Then, only the pure conjugate molecule was separated and purified by HPLC equipped with a C18 reversed-phase column and a H₂O/acetonitrile gradient eluent. Finally, the purified conjugate of Examples 4 to 6 was freeze-dried to obtain a red powder. The synthesis of the conjugate was identified by RP-HPLC and MALDI-TOF.

A prodrug nanoparticle solution was prepared by dispersing the conjugate in the form of the freeze-dried powder in distilled water (H₂O) or saline to a concentration of 1 mg/mL. The dispersion of prodrug nanoparticles was homogenized for 1 minute using a probe-type ultrasonic homogenizer, and analyzed by DLS.

The conjugate exists as self-assembled nanoparticles in a solution. Since the conjugate spontaneously forms nanoparticles in liquid, the terms conjugate and prodrug nanoparticles may be used interchangeably unless specified otherwise.

### Comparative Examples 1 and 2. Preparation of Cancer-Targeting Peptide

Peptide sequences cleaved by cathepsin B and having cancer-targeting ability were prepared for comparison with the examples. FRRG (SEQ ID NO: 13), known as a peptide cleaved by cathepsin B, and FRRLL (SEQ ID NO: 14), which was excluded during the selection of the cancer-targeting peptide of the present disclosure, were prepared. The peptides of SEQ ID NOS: 13 and 14 were acylated at the N-terminus through Fmoc solid-phase synthesis by Peptron Co., in the same manner as in Example 1.

### Comparative Examples 3 and 4. Preparation of Conjugate

Conjugates were prepared in the same manner as in Example 4, except that the cancer-targeting peptide of SEQ ID NO: 13 or 14 was used instead of the cancer-targeting peptide of SEQ ID NO: 1.

### TEST EXAMPLES

### Test Example 1. Confirmation of Synthesis of Conjugate and Analysis of Yield

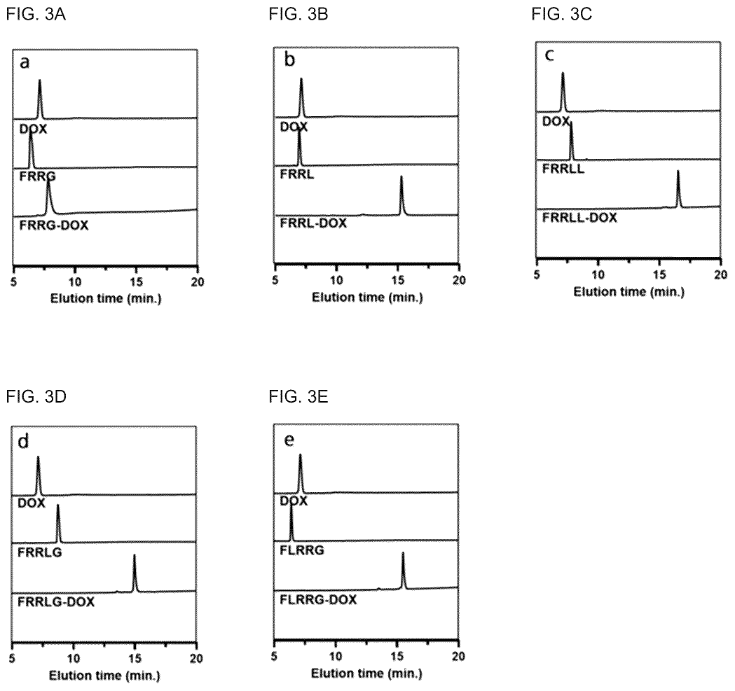

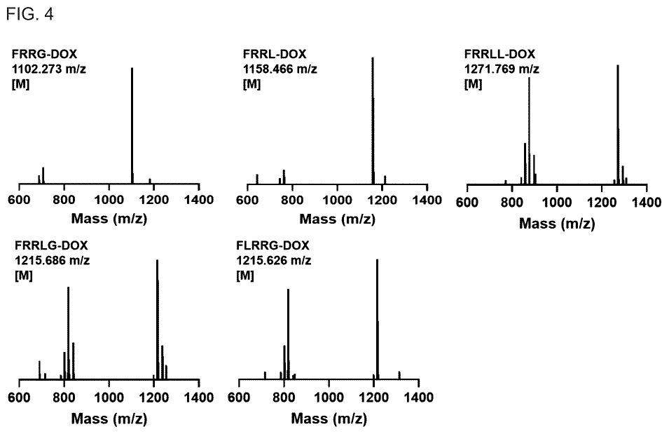

For the conjugates of Examples 4 to 6 and the conjugates of Comparative Examples 3 and 4, yield and purity were analyzed by HPLC and MALDI-TOF. The result is shown in Table 1, FIG. 3 and FIG. 4.

FIG. 3 shows the RP-HPLC chromatograms of the conjugates prepared in Examples 4 to 6 and Comparative Example 4 (b to e) and the conjugate prepared in Comparative Example 3 (a). They were analyzed by the gradient elution method (acetonitrile/H₂O=20:80 to 80:20, 25 min) using a C18 column (Eclipse XDB-C18, 4.6×150 mm, particle size=5 μm, Agilent Technologies).

TABLE 1

| | Comp. | | Comp. | | |
|---|---|---|---|---|---|
| | Ex. 3 | Ex. 4 | Ex. 4 | Ex. 5 | Ex. 6 |
| | (FRRG) | (FRRL) | (FRRLL) | (FRRLG) | (FLRRG) |
| Molar mass | 1102.273 | 1158.466 | 1271.769 | 1215.686 | 1215.626 |
| (m/z) | | | | | |
| Yield (%) | 78.0 | 83.9 | 82.9 | 94.9 | 92.9 |
| Purity (%) | 97.8 | 95.5 | 95.4 | 95.6 | 95.6 |

As shown in Table 1 and FIG. 3, although the conjugates prepared in Examples 4 to 6 and Comparative Example 4 were synthesized with high yield (82.9% to 94.9%) and purity (95.4% to 97.8%), the yield of the conjugate of Comparative Example 3 was below 80% (78%).

FIG. 4 shows the MALDI-TOF analysis result of the conjugates prepared in Examples 4 to 6, Comparative Example 3 and Comparative Example 4. CHCA (cyano-4-hydroxycinnamic acid) was used for the analysis (molecular weight range=500 to 2,000 Da).

As shown in Table 1 and FIG. 4, for the conjugates prepared in Examples 4 to 6, Comparative Example 3 and Comparative Example 4, the molecular weight of the conjugate corresponded exactly to the theoretical value: FRRG-DOX (Comparative Example 3); 1102.273 m/z [M], FRRL-DOX (Example 4); 1158.466 m/z [M], FRRLL-DOX (Comparative Example 4); 1271.769 m/z [M], FRRLG-DOX (Example 5); 1215.686 m/z [M], FLRRG-DOX (Example 6); 1215.626 m/z [M]).

To conclude, the conjugates of Examples 4 to 6 are advantageous in that they are very advantageous for mass production because the synthesis process is easy and yield and purity are high with no variation in the molecular weight.

### Test Example 2. Analysis of Size and Degree of Dispersion of Prodrug Nanoparticles

After preparing prodrug nanoparticles by dispersing the conjugates of Examples 4 to 6 and the conjugates of Comparative Examples 3 and 4 in distilled water or saline to a concentration of 1 mg/mL, they were analyzed by DLS.

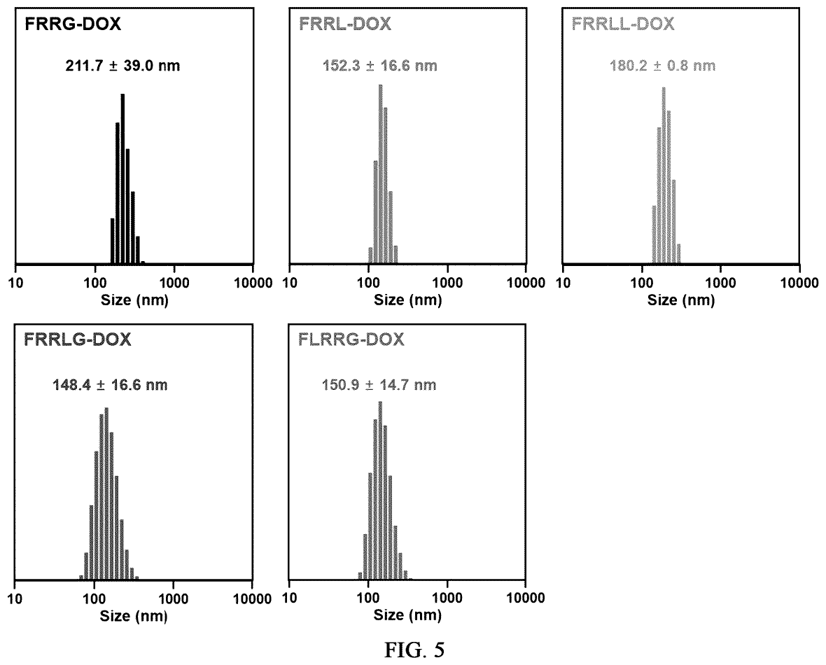

FIG. 5 shows the DLS analysis result for the prodrug nanoparticles of Examples 4 to 6 and the prodrug nanoparticles of Examples 3 and 4. Whereas the prodrug nanoparticles of Comparative Example 3 had an average diameter of 210 nm or larger, the prodrug nanoparticle of Examples 4 to 6 and Comparative Example 4 self-assembled into spherical nanoparticles with an average diameter of 150 to 180 nm due to the interaction between the anticancer agent (DOX) and the peptide (intermolecular π-π staking and hydrophobic interaction caused by amphiphilic characteristics). That is to say, when the conjugate self-assembles into prodrug nanoparticle, the shape and size are affected by the amphiphilicity and flexibility determined by the molecular structure of the conjugate.

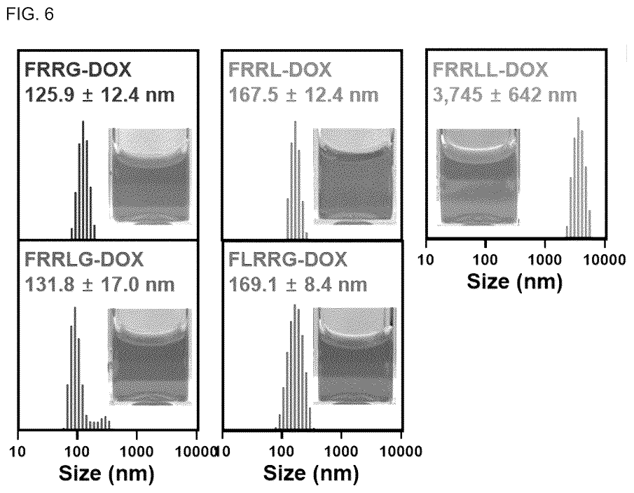

FIG. 6 shows the size and size distribution of the prodrug nanoparticles of Examples 4 to 6 and the prodrug nanoparticles of Examples 3 and 4 in saline. It was confirmed that the prodrug nanoparticles of Examples 4 to 6 maintained a size of 130 to 170 nm, except for the prodrug nanoparticles of Comparative Example 4 (FRRLL-DOX). The size of the prodrug nanoparticles of Comparative Example 3 was decreased by 59% to 125 nm.

It was confirmed that the prodrug nanoparticles of Comparative Example 4 have a size of 3,745±642 nm in saline. It is thought that they aggregated without maintaining their structure under the ionic liquid condition.

### Test Example 3. Long-Term Storability of Prodrug Nanoparticle

The prodrug nanoparticles of Examples 4 to 6 and the prodrug nanoparticles of Comparative Examples 3 to 4 were stored in saline for 75 hours to investigate storage stability. The change in the average diameter of the prodrug nanoparticles depending on storage time (0, 3, 6, 24, 48 and 72 hours) was analyzed by DLS.

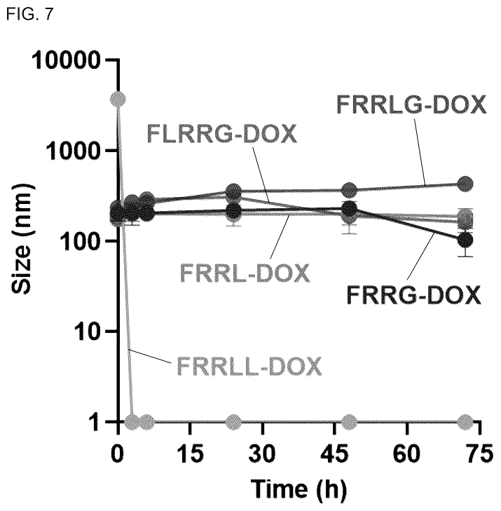

FIG. 7 shows the result of evaluating the colloidal stability of the prodrug nanoparticles prepared in Examples 4 to 6 and the prodrug nanoparticles prepared in Comparative Examples 3 to 4 in saline (mean±SD). It was confirmed that the prodrug nanoparticles of Examples 4 to 6 and the prodrug nanoparticles of Comparative Example 3 can stably maintain dispersibility up to 72 hours.

On the other hand, the average particle diameter of the prodrug nanoparticles of Comparative Example 3 began to decrease from 48 hours. However, they maintained dispersibility because aggregation was not observed until 72 hours.

The prodrug nanoparticles of Comparative Example 4 aggregated quickly to several micrometers in saline, and the measurement of diameter was impossible because they precipitated completely after 3 hours.

### Test Example 4. Proportion of Hydrophobic Surface of Prodrug Nanoparticles in Silico

The proportion of hydrophobic surface of the prodrug nanoparticles prepared in Examples 4 to 6 and the prodrug nanoparticles prepared in Comparative Example 3 was analyzed in silico. The dynamic simulation between conjugates constituting the prodrug nanoparticles and the evaluation of hydrophobicity were analyzed with the Desmond module of the Schrödinger suite using the following parameters: OPLS4; solvent model: TIP5P; ion placement: chloride; boundary conditions: orthorhombic box shape, box size calculation method(buffer); simulation time: 100 ns; approximate number of frames: 100; ensemble class: NPT; temperature: 300K; pressure: 1.01325 bar; thermostat method: Nose-Hoover chain; coulombic interaction cutoff radius: 9.0 Å.

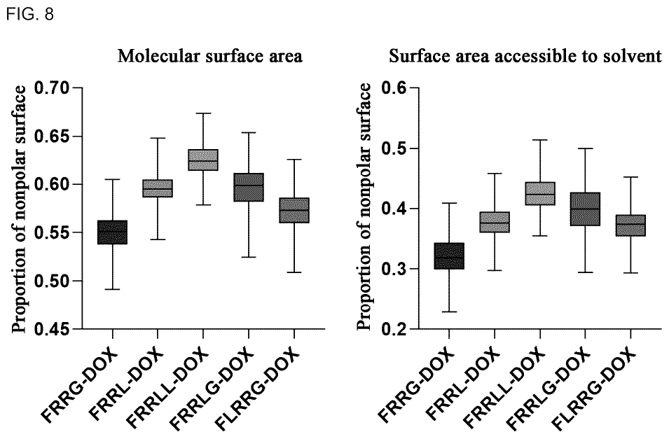

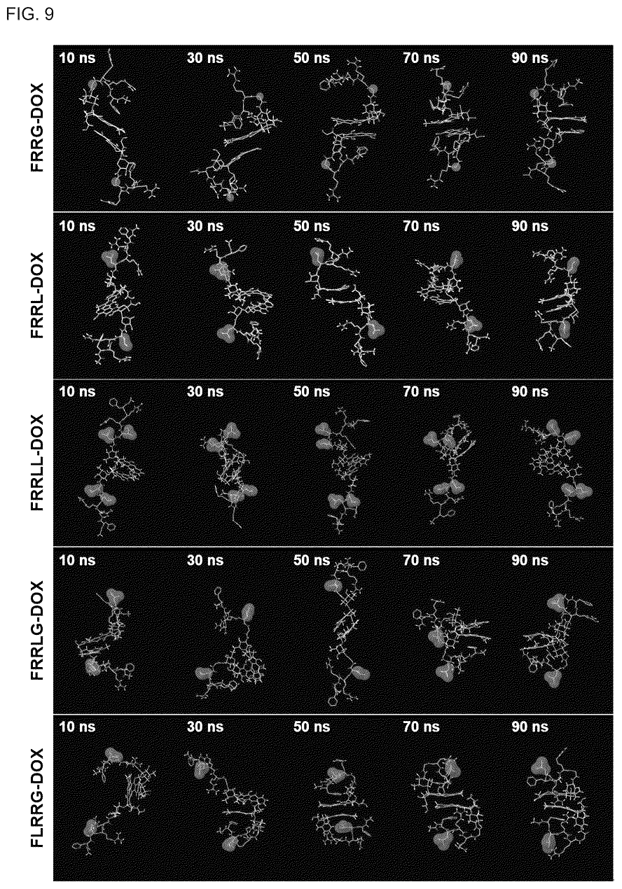

FIG. 8 shows the result of measuring the proportion of hydrophobic surface for the prodrug nanoparticles of Examples 4 to 6 and the prodrug nanoparticles of Comparative Examples 3 to 4. It was confirmed that the average surface area ratio of hydrophobic molecules do not exceed 60% for the prodrug nanoparticles of Examples 4 to 6, except for the prodrug nanoparticles of Comparative Example 4 (FRRLL-DOX). In addition, it was confirmed that the ratio of hydrophobic surface area accessible to the solvent was smaller than 40% for the prodrug nanoparticles of Examples 4 to 6. From these results, it can be seen that the prodrug nanoparticles of Examples 4 to 6 and Comparative Example 3 have an appropriate size of about 100 to 200 nm in a solution.

It was confirmed that the prodrug nanoparticles of Comparative Example 3 have the smallest average diameter in distilled water while having the lowest hydrophobicity. It is thought that the prodrug nanoparticles of Comparative Example 4 have the largest average diameter of several micrometers in saline since they have the highest hydrophobicity (ratio of molecular hydrophobic surface=62.5%, ratio of hydrophobic surface accessible to solvent=42.6%).

### Test Example 5. Analysis of Effect of Intermolecular Flexibility in Formation of Prodrug Nanoparticles in Silico

The proportion of hydrophobic surface of the prodrug nanoparticles of Examples 4 to 6 and the prodrug nanoparticles of Comparative Examples 3 to 4 was analyzed in silico. The dynamic simulation between conjugates constituting the prodrug nanoparticles and the evaluation of hydrophobicity were analyzed with the Desmond module of the Schrödinger suite using the following parameters: OPLS4; solvent model: TIP5P; ion placement: chloride; boundary conditions: orthorhombic box shape, box size calculation method (buffer); simulation time: 100 ns; approximate number of frames: 100; ensemble class: NPT; temperature: 300K; pressure: 1.01325 bar; thermostat method: Nose-Hoover chain; coulombic interaction cutoff radius: 9.0 Å.

FIG. 9 shows the intermolecular dynamic simulation result for the prodrug nanoparticles of Examples 4 to 6 and the prodrug nanoparticles of Comparative Examples 3 to 4.

As seen from FIG. 9, it was confirmed that the change in the molecular shape of the prodrug nanoparticles of Comparative Example 4 is minimized as compared to that of the prodrug nanoparticles of Examples 4 to 6 due to the rigidity of the Leu-Leu spacer. It is thought that the prodrug nanoparticles of Comparative Example 4 have a large average diameter of 1000 nm or greater due to low foldability.

The prodrug nanoparticles of Comparative Example 4 were excluded from the candidate materials in in-vivo and in-vitro experiments due to low dispersibility in physiological saline or a buffer such as PBS.

### Test Example 6. Structure of Prodrug Nanoparticles

The shape and size of the prodrug nanoparticles of Examples 4 to 6 and the prodrug nanoparticles of Comparative Example 3 were analyzed by TEM. For TEM analysis, a solution of the prodrug nanoparticles was placed on a copper grid, dried, and then negatively stained with 2% uracil acetate for 1.5 minutes.

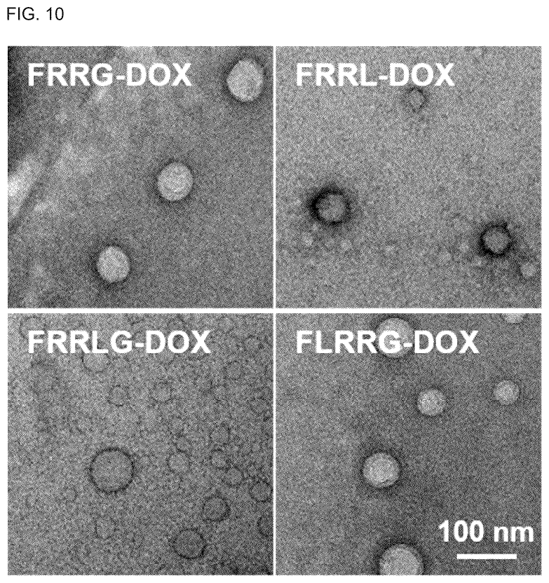

FIG. 10 shows the TEM images of the prodrug nanoparticles of Examples 4 to 6 and the prodrug nanoparticles of Comparative Example 3 (scale bar=200 nm). It was confirmed again that all the prodrug nanoparticles self-assemble to spherical particles in a solution. The prodrug nanoparticles according to the present disclosure exist as stable nanoparticle structures in a solution in the absence of a carrier material through intermolecular interaction.

### Test Example 7. Cathepsin B-Specific Degradation Activity of Prodrug Nanoparticles

It was investigated whether the prodrug nanoparticles of Examples 4 to 6 and the prodrug nanoparticles of Comparative Example 3 are degraded in the presence of the cathepsin B enzyme. First, after mixing 100 μM of the prodrug nanoparticles with 100 mM of a cathepsin B solution in a MES buffer (100 mM, pH 5.5), samples were taken depending on reaction time (0, 1, 3, 6, 9 and 24 hours) and observed by RP-HPLC. Specifically, they were analyzed by the gradient elution method (acetonitrile/H₂O=20:80 to 80:20, 25 min) using a C18 column (Eclipse XDB-C18, 4.6×150 mm, particle size=5 μm, Agilent Technologies).

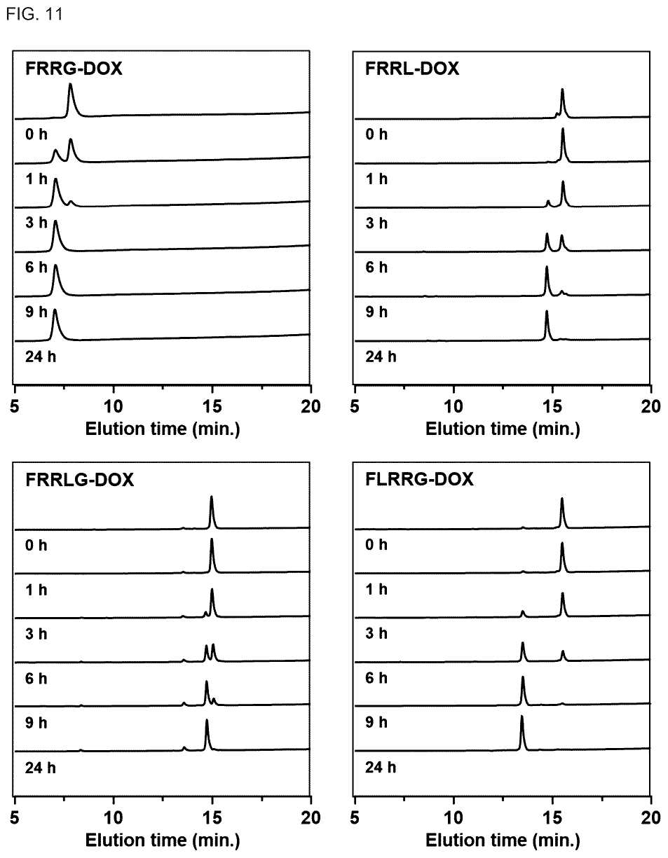

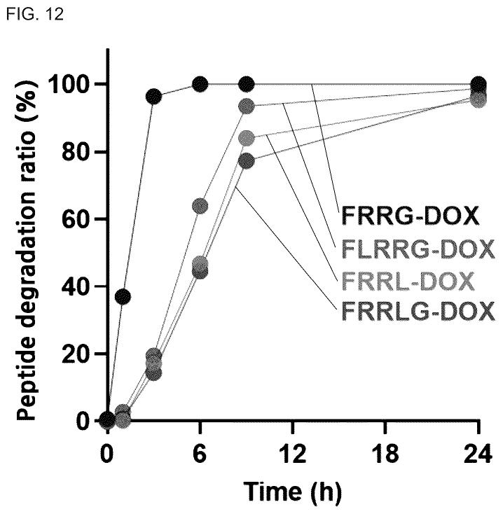

FIG. 11 shows the RP-HPLC chromatograms for the prodrug nanoparticles of Examples 4 to 6 and the prodrug nanoparticles of Comparative Example 3 in the 1 mM cathepsin B solution depending on time, and FIG. 12 shows the result of calculating peptide degradation ratio (%) by quantifying the RP-HPLC chromatograms in FIG. 11 depending on time.

As shown in FIG. 11, it was confirmed that the peaks corresponding to the prodrug nanoparticles (FRRG-DOX, FRRL-DOX, FRRLG-DOX, FLRRG-DOX) decrease and new peaks appear over reaction time. The newly appearing peaks did not match with the peaks of DOX, and did not match in between Examples 4-6 and Comparative Example 3.

This suggests that different degradation products are formed as the prodrug nanoparticles of Examples 4 to 6 and the prodrug nanoparticles of Comparative Example 3 are treated with the cathepsin B enzyme, depending on the peptide sequence (G-DOX, L-DOX and LG-DOX).

Specifically, for the prodrug nanoparticles of Example 4 (FRRL-DOX), a new peak of the FRRL-DOX conjugate appeared at 15.5 minutes, and a peak of L-DOX was observed at 14.7 minutes 3 hours after the reaction. The peak of the FRRL-DOX conjugate began to decrease and was not detected at all 24 hours after the reaction. The L-DOX was degraded again in cells and activated as DOX.

FIG. 12 shows the result of calculating peptide degradation ratio (%) from FIG. 11. The prodrug nanoparticles of Comparative Example 3 were degraded the most rapidly, and the prodrug nanoparticles of Examples 4 to 6 were also degraded fast and degraded completely with no remaining molecules after 24 hours.

It was confirmed that the prodrug nanoparticles according to the present disclosure is degraded effectively by the cathepsin B enzyme in saline too. In addition, it was further confirmed that the nanoparticles are not degraded by lysosome enzymes other than cathepsin B (The degradation ratio (%) was below 0-5% for Examples 4 to 6).

### Test Example 8. Cellular Uptake of Prodrug Nanoparticles

The cellular uptake and intranuclear localization of the prodrug nanoparticles (5 μM) were analyzed using HT29 colon cancer cells. In order to investigate the intracellular behavior and intracellular interaction of the prodrug nanoparticles, 2×10⁵ HT29 cells were seeded onto a glass-bottomed confocal dish. After stabilizing the cells by culturing for 24 hours, they were treated with the prodrug nanoparticles of Examples 4 to 6 and Comparative Example 3 (5 μM), and drug administration groups 1, 2 and 3 (FRRL-DOX, FRRLG-DOX, FLRRG-DOX) and a control group (FRRG-DOX) were obtained by incubating at 37° C. for 0, 24 and 48 hours. After the incubation was completed, the cells were washed with PBS, fixed with 4% paraformaldehyde for 15 minutes, and then stained with DAPI (4,6-diamidino-2-phenylindole) for 13 minutes. Each group was imaged by CLSM to obtain a fluorescence image, and fluorescence intensity was quantified from the fluorescence image to investigate the distribution of the prodrug nanoparticles using the Image-Pro software (Media Cybernetics, Rockville, MD, USA).

In order to further investigate whether the prodrug nanoparticles are degraded after cellular uptake, 1×10⁶ HT29 cells were seeded onto a 100-mm cell culture dish and stabilized by culturing for 24 hours. After administering the prodrug nanoparticles of Examples 4 to 6 and Comparative Example 3 (100 μM) to the cells, drug administration groups 1, 2 and 3 (FRRL-DOX, FRRLG-DOX, FLRRG-DOX) and a control group (FRRG-DOX) were obtained by incubating at 37° C. for 48 hours. Each group was washed with PBS and dispersed in distilled water (H₂O). The dispersion was filtered through a 0.45-μm syringe filter, and the recovered filtrate was subjected to mass analysis by MALDI-TOF.

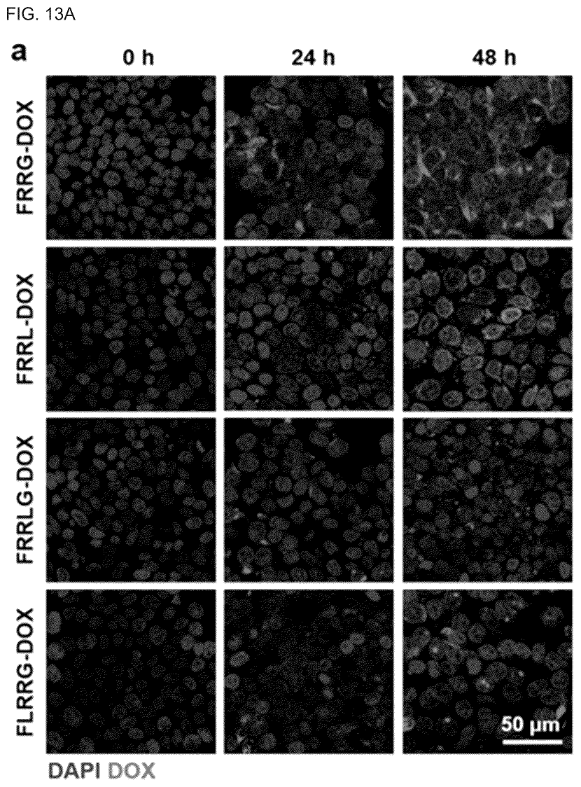

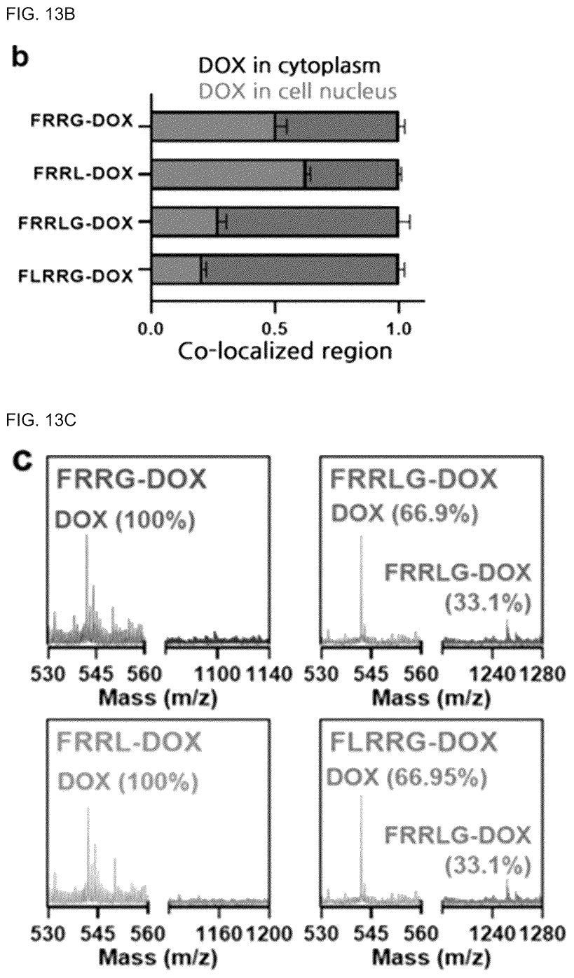

FIG. 13a shows the confocal microscopic images for the drug administration groups 1, 2 and 3 (FRRL-DOX, FRRLG-DOX, FLRRG-DOX) and the control group (FRRG-DOX) obtained after treating the HT29 cells with the prodrug nanoparticles of Examples 4 to 6 and the prodrug nanoparticles of Comparative Example 3.

As seen from FIG. 13a, the red fluorescence of the anticancer agent (DOX) began to be observed from the cancer cells from 24 hours after the cancer cells were treated with the prodrug nanoparticles of Examples 4 to 6 and the prodrug nanoparticles of Comparative Example 3. Through this, it can be seen that all of the prodrug nanoparticles of Examples 4 to 6 and the prodrug nanoparticles of Comparative Example 3 exhibit high cellular uptake of the anticancer agent.

For the prodrug nanoparticles of Example 5 and Example 6, (FRRLG-DOX and FLRRG-DOX), the red fluorescence (DOX) was observed mainly in the region other than the nucleus. For the prodrug nanoparticles of Example 4 (FRRL-DOX) and the prodrug nanoparticles of Comparative Example 3 (FRRG-DOX), the red fluorescence (DOX) was observed in the nucleus (blue region stained with DAPI).

FIG. 13b shows the result of comparing the fluorescence intensity of the anticancer agent (DOX) in the nucleus and cytoplasm from the confocal fluorescence images (48 h) of the drug administration groups 1, 2 and 3 (FRRL-DOX, FRRLG-DOX, FLRRG-DOX) and the control group (FRRG-DOX) obtained after treating the HT29 cells with the prodrug nanoparticles of Examples 4 to 6 and the prodrug nanoparticles of Comparative Example 3 (mean±SD, n=3).

As seen from FIG. 13b, for the prodrug nanoparticles of Example 5 and Example 6 (FRRLG-DOX and FLRRG-DOX), only 27% and 21% of the anticancer agent (DOX) was delivered into the nucleus, respectively. This result is 1.9 to 3.1 times lower as compared to the prodrug nanoparticles of Example 4 (FRRG-DOX).

It can be seen that, after the prodrug nanoparticles of Example 4 are introduced into the cytoplasm, they are degraded into the peptide and the anticancer agent (DOX) by cathepsin B present in cancer cells, and the degraded anticancer agent (DOX) is successfully delivered to the nucleus, like the prodrug nanoparticles of Comparative Example 3.

If the prodrug nanoparticles remain in the cells without being degraded, it is expected that the ratio of degradation by cathepsin B after endocytosis of the prodrug nanoparticles of Examples 5 and 6 will be low because they are not located in the nucleus.

Although the prodrug nanoparticles of Example 4 (FRRL-DOX) showed a slower rate of degradation by cathepsin B in vitro as compared to the prodrug nanoparticles of Comparative Example 3, the ratio of accumulation in the nucleus was 1.25 times higher as 63% for the prodrug nanoparticles of Example 4 (FRRL-DOX) as compared to the prodrug nanoparticles of Comparative Example 3 (FRRG-DOX). The increase of the ratio of accumulation in the nucleus by 5% or more means a significantly higher pharmacological effect for the same amount of the anticancer agent used, and means that the same effect can be achieved with 10% less amount of the anticancer agent.

Since the anticancer agent inevitably has side effects, the same effect achieved with a smaller amount is a remarkable effect in the art. In addition, the successful delivery of 60% or more of the anticancer agent present in the nanoparticles is also a significantly remarkable effect.

The prodrug nanoparticles according to the present disclosure were developed as a new peptide to solve the problems of FRRG, which is known as a tumor-specific peptide (insufficient pharmacological effect in vivo, and high accumulation in normal tissues other than cancer tissues). It was expected that binding ability with an anticancer agent can be increased and intermolecular flexibility and interaction can be improved through mutation, insertion and deletion of 1 to 2 amino acid residues. For this, the prodrug nanoparticles of Examples 4 to 6 were selected first in consideration of the formation of self-assembled nanoparticles, cleavage by cathepsin B, etc. It was confirmed from the result of in-silico experiments that the peptides of Examples 5 and 6 would provide better effect than the peptide of Example 4. However, in cell experiments in vitro, the prodrug nanoparticles of Example 4 exhibited better pharmacological effect than the prodrug nanoparticles of Examples 5 and 6, which could not be expected by those skilled in the art.

FIG. 13c shows the result of analyzing the anticancer agent (DOX) from the filtrates of the drug administration groups 1, 2 and 3 (FRRL-DOX, FRRLG-DOX, FLRRG-DOX) and the control group (FRRG-DOX) obtained after treating the HT29 cells with the prodrug nanoparticles of Examples 4 to 6 and the prodrug nanoparticles of Comparative Example 3 by MALDI-TOF.

As seen from FIG. 13c, it was confirmed that the anticancer agent (DOX) was successfully separated from the prodrug nanoparticles of Examples 4 to 6 and the prodrug nanoparticles of Comparative Example 3 in the cancer cells.

For the drug administration groups 2 and 3 (FRRLG-DOX, FLRRG-DOX), the prodrug nanoparticles of Examples 5 to 6 were identified in the cancer cells. However, for the drug administration group 1 (FRRL-DOX) and the control group (FRRG-DOX), the prodrug nanoparticles of Example 4 and the prodrug nanoparticles of Comparative Example 3 were not observed, and only the anticancer agent (DOX) was observed. That is, it can be seen that the prodrug nanoparticles of Example 4 and the prodrug nanoparticles of Comparative Example 3 were completely degraded in the cancer cells. However, the prodrug nanoparticles of Examples 5 and 6 exhibited slightly low sensitivity to cathepsin B in the cancer cells.

In addition, although it was expected that the prodrug nanoparticles of Examples 4 to 6 and the prodrug nanoparticles of Comparative Example 3 would be degraded by the cathepsin B enzyme upon reaction with cancer cells and produce fragment-DOX (G-DOX, L-DOX and LG-DOX), the peak of the fragment-DOX was not observed in the cells. Through this, it can be seen that, although the fragment-DOX is produced from the reaction of the prodrug nanoparticles of Examples 4 to 6 and the prodrug nanoparticles of Comparative Example 3 with the cathepsin B enzyme, it is immediately degraded by lysosomes in the cells to produce DOX, which is delivered into the nucleus and effectively exhibits anticancer effect.

### Test Example 9. Cancer Cell Specificity and Cytotoxicity of Prodrug Nanoparticles

The cancer cell-specific efficacy and cytotoxicity of the prodrug nanoparticles according to the present disclosure were analyzed. Because the side effects of the anticancer agent (DOX) occur mainly in the heart, H9C2 cells (cardiomyocytes) were used as normal cells.

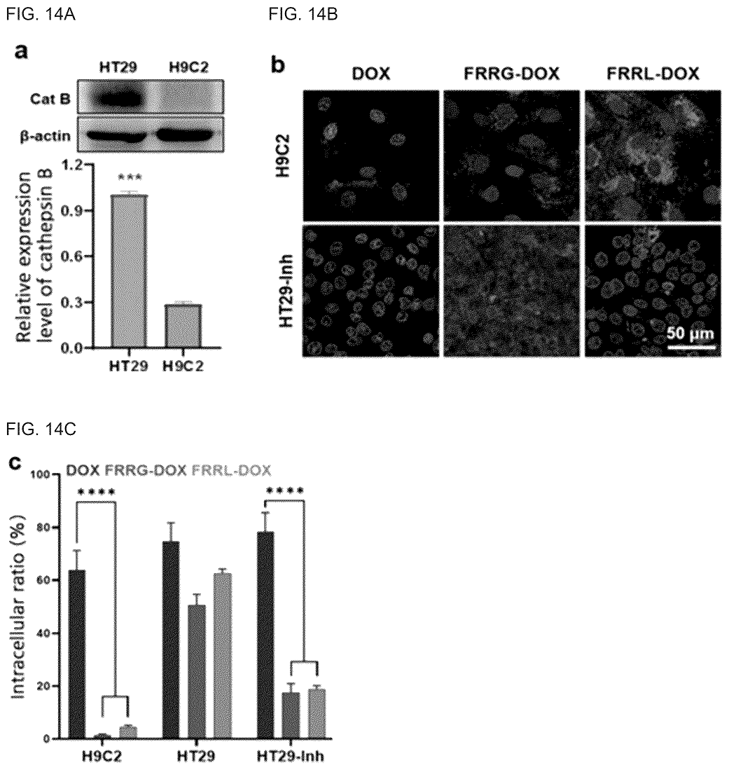

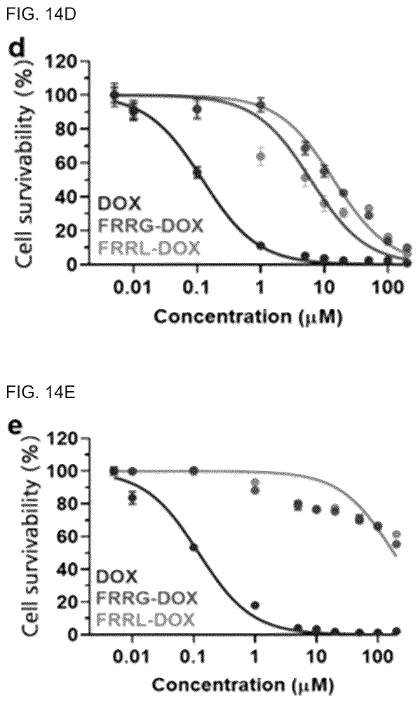

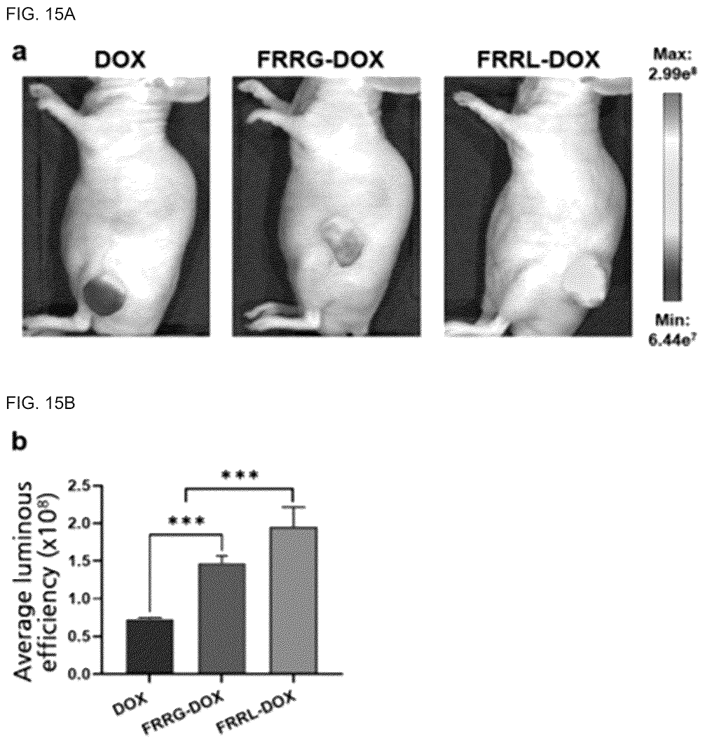

Western blot was performed to compare the expression level of cathepsin B in HT29 cells and H9C2 cells. The result is shown in FIG. 14a. Specifically, 1×10⁶ HT29 cells and 1×10⁶ H9C2 cells were seeded onto a 100 mm cell culture dish, respectively, stabilized for 24 hours, washed with PBS, and then dispersed in a lysis buffer containing 1% protease inhibitors. The obtained lysate was centrifuged at 12,000 rpm for 25 minutes to remove cell debris. After recovering the supernatant, the concentration of proteins was quantitated using a BCA kit. The supernatant was separated by SDS-PAGE (sodium dodecyl sulfate-polyacrylamide gel electrophoresis) on a 10% gel, transferred to a PVDF (polyvinylidenedifluoride) separation membrane, and then incubated with TBS-T (Tween 20-including Tris-buffered saline) containing 5% BSA (bovine serum albumin) for 2 hours in order to block binding with IgG (non-specific immunoglobulin G). Then, after treating with goat anti-mouse cathepsin B primary antibodies (500:1) at 4° C. for 24 hours and washing 3 times with TBS-T, followed by incubation with HRP-conjugated mouse anti-goat IgG antibodies for 2 hours, immunoresponsive bands were detected using an ECL (enhanced chemiluminescence) system.

Then, experiment was performed as follows to analyze specific activity for cells. First, 2×10⁵ HT29 cells and 2×10⁵ H9C2 cells were seeded onto a glass-bottomed confocal dish, respectively. Cathepsin B-inhibited cells (HT29-Inh) were prepared by pretreating HT29 cells with Z-Phe-Ala-FMK (20 μM) for 24 hours. After stabilizing the cells by culturing for 24 hours, and administering the anticancer agent (DOX) alone (5 μM) or together with the prodrug nanoparticles of Example 4 and Comparative Example 3 (5 μM), respectively, a single administration group, drug administration groups and a control group were obtained by incubating at 37° C. for 48 hours. After the incubation was completed, the cells were washed with PBS, fixed with 4% paraformaldehyde for 15 minutes, and then stained with DAPI (4,6-diamidino-2-phenylindole) for 13 minutes. Each group was imaged by CLSM to obtain a fluorescence image, and fluorescence intensity was quantified from the fluorescence image to investigate the distribution of the prodrug nanoparticles using the Image-Pro software (Media Cybernetics, Rockville, MD, USA).

In addition, cytotoxicity was investigated using CCK-8 (cell counting kit-8). Specifically, 1×10⁴ HT29 cells and 0.5×10⁴ H9C2 cells were seeded onto a 96-well plate, respectively, and stabilized by culturing for 24 hours. A single administration group, drug administration groups and a control group were obtained by administering the anticancer agent (DOX) alone (0.01 to 100 μM) or together with the prodrug nanoparticles of Example 4 and Comparative Example 3 (0.01 to 100 μM), respectively, to each well and incubating at 37° C. for 48 hours. After the incubation was completed, a culture medium containing a 10% CCK-8 solution was added. After conducting reaction for 20 minutes, analysis was made with a microplate reader at a wavelength of 450 nm. After plotting cell survivability depending on concentration, IC₅₀ (half-maximal inhibitory concentration) values were obtained for the single administration group, drug administration groups and control group using the GraphPad Prism 8 software (GraphPad Software, San Diego, CA, USA).

### Statistical Analysis

For statistical analysis, the significant difference between the groups were tested by Student's t-test. The comparison between two or more experimental groups was performed by Tukey-Kramer post-hoc test and analysis of variance (ANOVA). P values smaller than 0.05 were indicated by *, p values smaller than 0.01 by **, p values smaller than 0.001 by ***, and p values smaller than 0.0001 by ****. The values were presented as mean±standard deviation (SD).

FIG. 14a shows the result of analyzing the expression level of cathepsin B in the HT29 cells and H9C2 cells by western blot (top) and the result of quantifying band intensities (bottom). As seen from FIG. 14a, the HT29 cells expressed cathepsin B by 3.5 times more than the H9C2 cells. Accordingly, it can be seen that the cathepsin B enzyme is enough to be a target for cancer cells.

FIG. 14b shows the confocal microscopic images of the single administration group (DOX), the drug administration group (Example 4; FRRL-DOX) and the control group (Comparative Example 3; FRRG-DOX) for the H9C2 cells and HT29-Inh cells.

As seen from FIG. 14b, in the H9C2 cells and HT29-Inh cells, the fluorescence of anticancer agent (DOX) was observed only in the cytoplasm for the drug administration group (Example 4; FRRL-DOX) (5 μM) and the control group (Comparative Example 3; FRRG-DOX). That is to say, since the prodrug nanoparticles of Example 4 and Comparative Example exist in inactive state not expressing the cathepsin B enzyme in the H9C2 cells and HT29-Inh cells, they did not penetrate into the nucleus but were observed only in the cytoplasm. In contrast, for the single administration group (DOX), the prodrug nanoparticles penetrated into the cell nucleus and induced cell death, regardless of the presence or absence of the cathepsin B enzyme.

FIG. 14c shows the result of quantifying the fluorescence intensity of the anticancer agent (DOX) measured from the images of FIG. 14b. It can be seen that the prodrug nanoparticles according to the present disclosure (Example 4) exhibited the activity of targeting malignant tumors. In contrast, it can be seen that the anticancer agent (DOX) penetrates into the nucleus of normal cells without tumor specific and exhibits toxicity.

The ratio of accumulation of the prodrug nanoparticles of Example 4 (FRRL-DOX) in the nucleus of the H9C2 and HT29-Inh cells was measured as 5% and 19%, respectively, suggesting that they hardly induce toxicity for normal cells.

FIG. 14d shows the result of measuring the cell survivability of HT29 cells for the single administration group a (DOX), the drug administration group (Example 4; FRRL-DOX) and the control group (Comparative Example 3; FRRG-DOX) depending on concentration. FIG. 14e shows the result of measuring the cell survivability of H9C2 cells for the single administration group (DOX), the drug administration group (Example 4; FRRL-DOX) and the control group (Comparative Example 3; FRRG-DOX) depending on concentration. Data were presented as mean±SD (n=3), and significant difference was indicated by *** (p<0.001) and **** (p<0.0001). In addition, the IC₅₀ values obtained from the result of FIG. 14d and FIG. 14e are given in Table 2.

TABLE 2

| | | Example 4 | Comparative Example 3 |
|---|---|---|---|
| | DOX | (FRRL-DOX) | (FRRG-DOX) |
| IC₅₀ (HT29) | 0.12 | 6.13 | 14.07 |
| IC₅₀ (H9C2) | 0.12 | 182.4 | 158.2 |

From FIG. 14d, FIG. 14e and Table 2, it can be seen that the drug administration group (Example 4; FRRL-DOX) shows superior efficacy of cell death for cancer cells at 1 to 100 μM (survival rate: 0%) and low efficacy of cell death for normal cells (survival rate: 80%), demonstrating that they exhibit high anticancer effect and few side effects for normal cells.

In contrast, the control group (Comparative Example 3; FRRG-DOX) showed low efficacy of cell death for cancer cells at 1 μM, demonstrating that the prodrug nanoparticles of Example 4 exhibit higher anticancer effect than the prodrug nanoparticles of Comparative Example 3.

The single administration group (DOX) showed no difference in the survival rate in the tumor cells and the normal cells, suggesting the high risk of side effects since it kills normal cells as well at the concentration where it kills the cancer cells.

As seen from Table 2, the IC₅₀ value of the prodrug nanoparticles of Example 4 was 6.13 μM for the HT29 cells, and 182.4 μM for the H9C2 cells. That is to say, it was demonstrated that the prodrug nanoparticles of Example 4 exhibit higher toxicity for cancer cells and lower toxicity for normal cells as compared to the prodrug nanoparticles of Comparative Example 3. In other words, it can be seen that the prodrug nanoparticles according to the present disclosure exhibits significantly superior pharmacological effect as compared to the prodrug nanoparticles of Comparative Example 3.

Meanwhile, when the anticancer agent (DOX) was used alone, the IC₅₀ value was 0.12 μM for both the HT29 and H9C2 cells, suggesting severe side effects.

### Test Example 10. Distribution of Prodrug Nanoparticles In Vivo

The in-vivo behavior of the prodrug nanoparticles according to the present disclosure was investigated.

### Animal Model of Cancer (Tumor-Bearing Mice)

Experimental mice were housed in a pathogen-free facility of the Korea Institute of Science and Technology (KIST) with 12-hour light/dark cycles, with free access to feed and water. 5-week-old male BALB/c nu/nu mice purchased from NaraBio were used for experiment after one week of adaptation.

1×10⁷ HT29 cells were subcutaneously injected into the left flank of the 5-week-old male BALB/c nu/nu mice. An animal model of cancer was prepared by breeding the mice until the tumor size grew to about 100 to 200 mm³.

### Drug Administration

Test groups 1 to 5 were prepared by injecting an anticancer agent (DOX) (4 mg/kg) and the prodrug nanoparticles of Example 4 or Comparative Example 3 (4 or 10 mg/kg based on the concentration of DOX) to the animal model of cancer via the tail vein (n=2 for each test group).

After 3, 6 and 9 hours of the drug injection, the test groups were imaged using an IVIS Lumina Series III system. After euthanasia, liver, lung, spleen, kidney, heart and tumor tissues were recovered.

The obtained tissues were sliced into 10 μm-thick sections, which were mounted individually on a slide glass, and washed tice with DPBS. Then, after staining with DA for 20 minutes, fluorescence signals were observed by CLSM. All the animal experiments were performed in compliance with the relevant laws and institutional guidelines of the Institutional Animal Care and Use Committee (IACUC, approval no. 2021-143) of the KIST.

TABLE 3

| | Treatment |
|---|---|
| Test group 1 | Animal model of cancer −> intravenous administration of |
| (DOX) | 4 mg/kg anticancer agent (DOX) alone |
| Test group 2 | Animal model of cancer −> intravenous administration of |
| (FRRG-DOX) | prodrug nanoparticles of Comparative Example 3 (4 |
| (4 mg/kg) | mg/kg based on DOX concentration) |
| Test group 3 | Animal model of cancer −> intravenous administration of |
| (FRRG-DOX) | prodrug nanoparticles of Comparative Example 3 (10 |
| (10 mg/kg) | mg/kg based on DOX concentration) |
| Test group 4 | Animal model of cancer −> intravenous administration of |
| (FRRL-DOX) | prodrug nanoparticles of Example 4 (4 mg/kg based on |
| (4 mg/kg) | DOX concentration) |
| Test group 5 | Animal model of cancer −> intravenous administration of |
| (FRRL-DOX) | prodrug nanoparticles of Example 4 (10 mg/kg based on |
| (10 mg/kg) | DOX concentration) |

### Statistical Analysis

For statistical analysis, the significant difference between the groups were tested by Student's t-test. The comparison between two or more experimental groups was performed by Tukey-Kramer post-hoc test and analysis of variance (ANOVA). P values smaller than 0.05 were indicated by *, p values smaller than 0.01 by **, p values smaller than 0.001 by ***, and p values smaller than 0.0001 by ****. The values were presented as mean±standard deviation (SD).

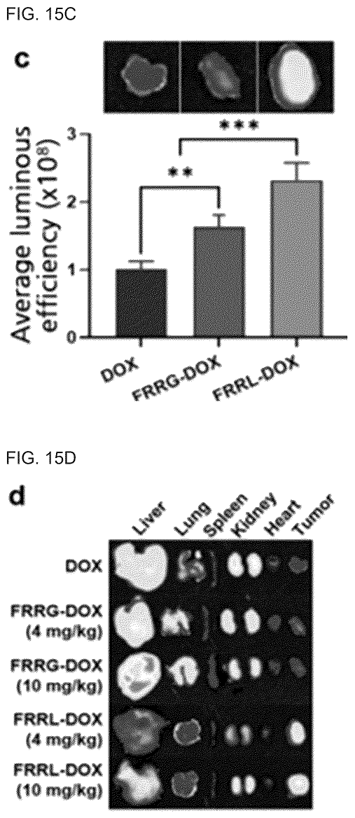

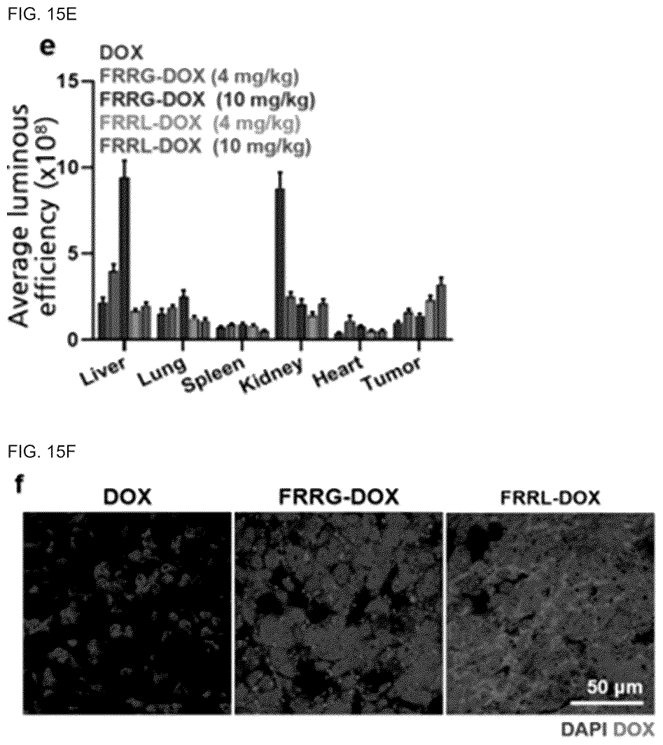

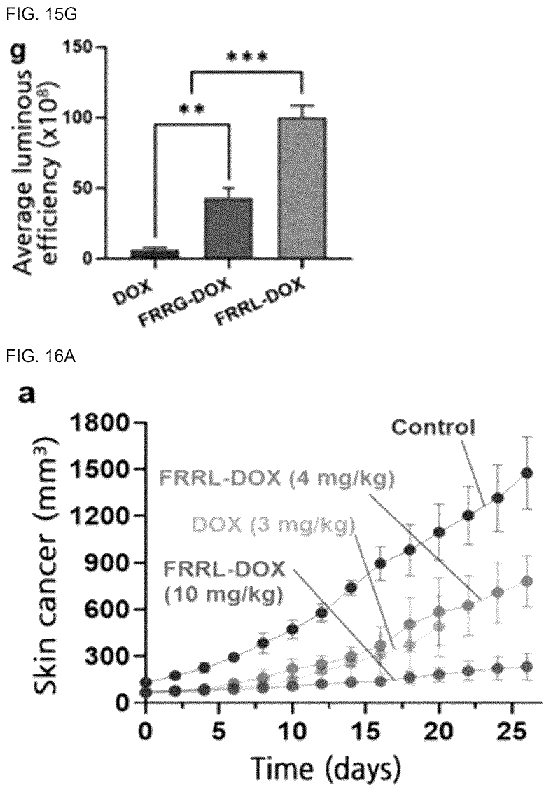

FIG. 15a shows the digital fluorescence microscopic images for the test groups 1, 2 and 4 obtained 9 hours after injection of the anticancer agent (DOX) (4 mg/kg) or the prodrug nanoparticles of Example 4 or Comparative Example 3 (4 mg/kg based on DOX concentration) to the animal model of cancer. FIG. 15b shows the fluorescence intensity of cancer tissue quantitated from the images of FIG. 15a.

As seen from FIG. 15a and FIG. 15b, the prodrug nanoparticles of Example 4 exhibits significantly high accumulation efficiency for cancer tissues as compared to the anticancer agent (DOX) alone or the prodrug nanoparticles of Comparative Example 3. The prodrug nanoparticles of Example 4 exhibits not only significantly superior cancer-targeting ability but also remarkably superior accumulation efficiency for cancer as compared to the existing prodrug nanoparticles (Comparative Example 3).

FIG. 15c shows the result of isolating cancer tissue from the test groups 1, 2 and 4 and analyzing fluorescence intensity. The prodrug nanoparticles of Example 4 showed 2.3 times and 1.4 times higher cancer-targeting ability, respectively, as compared to the anticancer agent (DOX) and the prodrug nanoparticles of Comparative Example 3.

In the foregoing experiment, the prodrug nanoparticles of Comparative Example 3 showed lower dispersion stability in saline in the long term as compared to the prodrug nanoparticles of Example 4. Although the prodrug nanoparticles of Example 4 and Comparative Example 3 are different in only one peptide sequence, the prodrug nanoparticles of Example 4 show remarkably superior pharmacological effect than the existing prodrug nanoparticles (Comparative Example 3) because of the difference in the shape, intermolecular interaction, etc. of the prodrug nanoparticles.

FIG. 15d shows the fluorescence microscopic images of the tissues of major organs (liver, lung, spleen, kidney, heart and tumor) from the test group 1 to 5, and FIG. 15e shows the result of isolating the tissues of major organs (liver, lung, spleen, kidney, heart and tumor) from the test group 1 to 5 and analyzing fluorescence intensity.

As seen from FIG. 15d and FIG. 15e, the distribution of the prodrug nanoparticles in the major organs in vivo was identified. For the test group 1 (DOX), wherein the anticancer agent was administered alone, the anticancer agent was accumulated mostly in the kidney. The fluorescence intensity was 8.8 times higher in the kidney tissues than in the cancer tissues.

In contrast, for the test groups 4 and 5, wherein the prodrug nanoparticles of Example 4 were administered, the amount of accumulation was 2 times or more in the cancer tissues than in the kidney tissues.

For the test groups 2 and 3, wherein the prodrug nanoparticles of Comparative Example 3 were administered, the anticancer agent was accumulated significantly more in the liver tissues than in the cancer tissues (2.4 times and 6.7 times). The excessive accumulation of the prodrug nanoparticles of Comparative Example 3 in the normal cells of the liver, rather than in cancer tissues, may cause side effects such as acute hepatoxicity.

In addition, the prodrug nanoparticles of Comparative Example 3 exist unstably in the blood and facilitate opsonization and liver uptake. Accordingly, the prodrug nanoparticles of Example 4, which exhibit superior stability in vivo, exhibit significantly high targeting efficiency for cancer tissues as compared to other normal tissues, and exhibit 2.4 times and 4.8 times lower accumulation efficiency for liver tissues than the prodrug nanoparticles of Comparative Example 3, are the most suitable for a pharmaceutical composition for preventing or treating cancer.

FIG. 15f shows the confocal microscopic images of cancer tissues isolated from the test groups 1, 2 and 4 obtained after staining with DAPI. FIG. 15g shows the result of quantifying the fluorescence intensity of the anticancer agent (DOX) in cancer tissues isolated from the test groups 1, 2 and 4.

As seen from FIG. 15f and FIG. 15g, red fluorescence (DOX) was hardly observed from the cancer tissues of the test group 1 (DOX) to which the anticancer agent was administered alone. However, for the test group 4 to which the prodrug nanoparticles of Example 4 were administered, red fluorescence significantly stronger by 6.7 times and 2.6 times, respectively, was observed in the cancer tissues as compared to the test group 1 and the test group 3.

### Test Example 11. Analysis of Anticancer Effect of Prodrug Nanoparticles

### Animal Model of Cancer (Tumor-Bearing Mice)

Experimental mice were housed in a pathogen-free facility of the Korea Institute of Science and Technology (KIST) with 12-hour light/dark cycles, with free access to feed and water. 5-week-old male BALB/c nu/nu mice purchased from NaraBio were used for experiment after one week of adaptation.

1×10⁷ HT29 cells were subcutaneously injected into the left flank of the 5-week-old male BALB/c nu/nu mice. An animal model of cancer was prepared by breeding the mice until the tumor size grew to about 60 to 80 mm³.

### Drug Administration

Test groups 6 to 8 were prepared by injecting an anticancer agent (DOX) (3 mg/kg) or the prodrug nanoparticles of Example 4 (4 or 10 mg/kg based on DOX concentration) to the animal model of cancer via the tail vein once in 3 days at the same time (n=5 for each test group). The overall treatment period was 26 days, and the number of drug administration was 8 times.

After the treatment period, tumor volume, body weight and survival rate of each test group were measured every 2 days. After euthanasia, liver, lung, spleen, kidney, heart and cancer tissues were recovered and subjected to histological and TUNEL assay.

TABLE 4

| | Treatment |
|---|---|
| Animal model | Animal model of cancer −> intravenous administration |
| of cancer | of 4 mg/kg saline once in 3 days (8 times in total) |
| (control) | |
| Test group 6 | Animal model of cancer −> intravenous administration |
| (DOX) | of 4 mg/kg anticancer agent (DOX) alone once in 3 days |
| | (8 times in total) |
| Test group 7 | Animal model of cancer −> intravenous administration |
| (FRRL-DOX) | of prodrug nanoparticles of Example 4 (4 mg/kg based |
| (4 mg/kg) | on DOX concentration) once in 3 days (8 times in total) |
| Test group 8 | Animal model of cancer −> intravenous administration |
| (FRRL-DOX) | of prodrug nanoparticles of Example 4 (10 mg/kg based |
| (10 mg/kg) | on DOX concentration) once in 3 days (8 times in total) |

The tumor volume was calculated from the longest diameter×(the shortest diameter)²×0.53 of the cancer tissue, and a tumor volume larger than 1,000 mm³ was judged as death.

For histological analysis, each of the recovered liver, lung, spleen, kidney, heart and cancer tissues was fixed with 4% paraformaldehyde for 15 minutes, embedded in wax, and then sliced to 10 μm-thick sections. Then, after staining with H&E, the morphological damage to the tissues was analyzed by imaging with an optical microscope.

The cancer tissue was stained by TUNEL (terminal deoxynucleotidyl transferase dUTP nick end labeling) analysis for visualization of the apoptotic region.

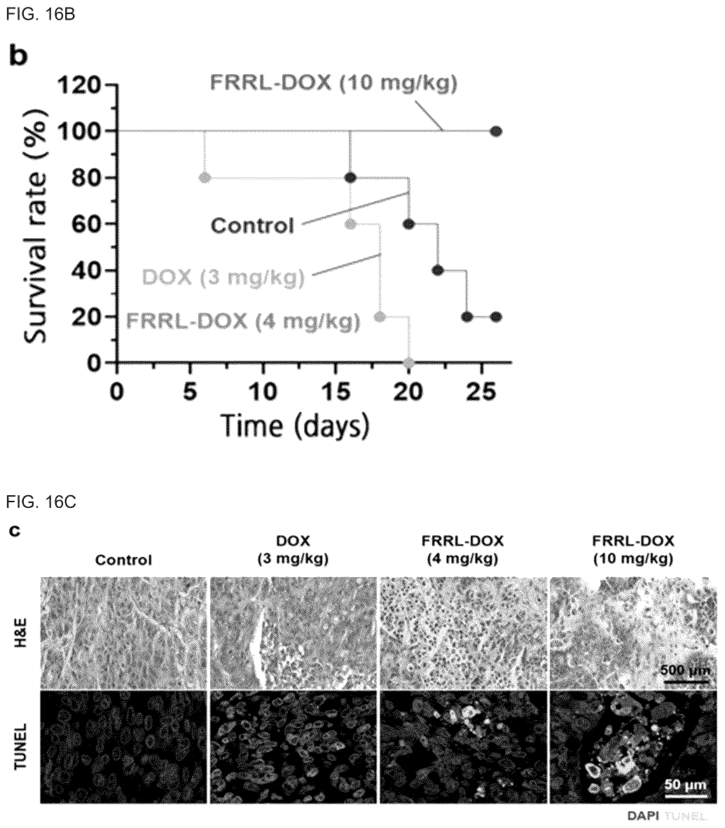

FIG. 16a shows the result of measuring the tumor volume for the test groups 6 to 8 depending on time. FIG. 16b shows the result of measuring survival rate for the test groups 6 to 8 depending on time. FIG. 16c shows the TUNEL assay and histopathological analysis results for cancer tissues isolated from the test groups 6 to 8.

As seen from FIG. 16, for the test group 6 to which the anticancer agent was administered alone (DOX), death was observed within 5 days due to severe systemic toxicity and all the mice died within 20 days. For the animal model of cancer (Control), 80% or more of the mice died. In contrast, the test groups 7 and 8 to which the prodrug nanoparticles of Example 4 were administered showed a survival rate of 100% even after 26 days.

Tumor growth was inhibited significantly for the test groups 7 and 8, with a tumor volume smaller by 1.9 to 6.3 times than the tumor volume of the test group 6. In addition, since no severe systemic toxicity was induced for normal tissues other than the cancer tissues, the prodrug nanoparticles of Example 4 are very useful for a composition for preventing or treating cancer.

As seen from FIG. 16c, significant anti-tumor activity was conformed for the test groups 7 and 8 to which the prodrug nanoparticles of Example 4 were administered, with the morphology of the cancer tissue completely destroyed. In contrast, for the test group 6 to which the anticancer agent was administered alone (DOX), relatively weak damage of the cancer tissue was observed.

TUNEL visualizes DNA fragmentation in apoptosis with green fluorescence. As a result of TUNEL assay, green fluorescence was not observed in the cancer tissue of the animal model of cancer (Control), and weak green fluorescence was observed only in a part of the cancer tissue of the test group 6 to which the anticancer agent was administered alone (DOX). In the cancer tissues of the test groups 7 and 8 to which the prodrug nanoparticles of Example 4 were administered, strong green fluorescence was observed. That is to say, it can be seen that the prodrug nanoparticles of Example 4 substantially induce the death of cancer cells as compared to when the anticancer agent is used alone.

### Test Example 12. Comparison of Toxicity of Prodrug Nanoparticles In Vivo Depending on Administration Method

The side effects of the prodrug nanoparticles in vivo depending on administration method was investigated.

### Single Administration

Test groups 9 to 12 were prepared by injecting an anticancer agent (DOX) (3 or 4 mg/kg) or the prodrug nanoparticles of Example 4 (4 or 10 mg/kg based on DOX concentration) once to tumor-free BALB/c mice (5-week-old) via the tail vein (n=5 for each test group).

TABLE 5

| | Treatment |
|---|---|
| Normal group | Normal animal model |
| (control) | |
| Test group 9 | Normal animal model −> intravenous administration |
| (DOX 3 mg/kg) | of 3 mg/kg anticancer agent (DOX) alone once |
| Test group 10 | Normal animal model −> intravenous administration |
| (DOX, 4 mg/kg) | of 4 mg/kg anticancer agent (DOX) alone once |
| Test group 11 | Normal animal model −> intravenous administration |
| (FRRL-DOX) | of prodrug nanoparticles of Example 4 (4 mg/kg |
| (4 mg/kg) | based on DOX concentration) once |
| Test group 12 | Normal animal model −> intravenous administration |
| (FRRL-DOX) | of prodrug nanoparticles of Example 4 (10 mg/kg |
| (10 mg/kg) | based on DOX concentration) once |

### Multiple Administration

Test groups 13 to 16 were prepared by injecting an anticancer agent (DOX) (3 or 4 mg/kg) or the prodrug nanoparticles of Example 4 (4 or 10 mg/kg based on DOX concentration) 8 times in total for 26 days (once in 3 days) to tumor-free BALB/c mice (5-week-old) via the tail vein (n=5 for each test group).

TABLE 6

| | Treatment |
|---|---|
| Normal group | Normal animal model |
| (control) | |
| Test group 13 | Normal animal model −> intravenous |
| (DOX 3 mg/kg) | administration of 3 mg/kg anticancer agent (DOX) |
| | alone once in 3 days (8 times in total) |
| Test group 14 | Normal animal model −> intravenous |
| (DOX, 4 mg/kg) | administration of 4 mg/kg anticancer agent (DOX) |
| | alone once in 3 days (8 times in total) |
| Test group 15 | Normal animal model −> intravenous |
| (FRRL-DOX) | administration of prodrug nanoparticles of |
| (4 mg/kg) | Example 4 (4 mg/kg based on DOX concentration) |
| | once in 3 days (8 times in total) |
| Test group 16 | Normal animal model −> intravenous |
| (FRRL-DOX) | administration of prodrug nanoparticles of |
| (10 mg/kg) | Example 4 (10 mg/kg based on DOX |
| | concentration) once in 3 days (8 times in total) |

### Analysis Method

Tumor volume, body weight and survival rate were measured every day after injection of the drug to the test groups. 8 days later, the mice were euthanized and liver, lung, spleen, kidney, heart and cancer tissues were recovered. Each of the recovered liver, lung, spleen, kidney, heart and cancer tissues was fixed with 4% paraformaldehyde for 15 minutes, embedded in wax, and then sliced to 10 μm-thick sections. Then, after staining with H&E, the morphological damage to the tissues was analyzed by imaging with an optical microscope. All the animal experiments were performed in compliance with the relevant laws and institutional guidelines of the Institutional Animal Care and Use Committee (IACUC, approval no. 2021-143) of the KIST.

For evaluation of hematotoxicity, 1 mL of blood was taken from each test group, and blood plasma was separated by centrifuging at 4,500 rpm for 20 minutes. Blood analysis was performed according to the instructions of KNOTUS Co., Ltd. (Incheon, Korea).

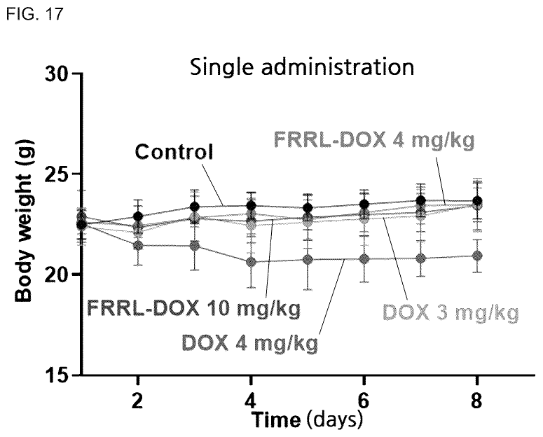

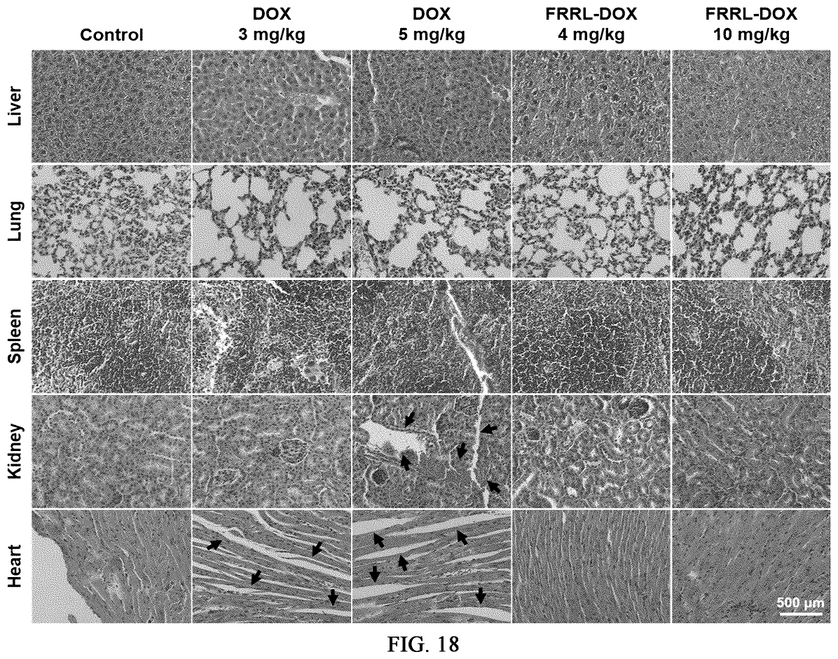

FIG. 17 shows the result of measuring body weight change for the normal group and the test groups 9 to 12. FIG. 18 shows the histochemical analysis result of the liver, lung, spleen, kidney and heart tissues of the normal group and the test groups 9 to 12 stained with H&E.

As seen from FIG. 17, no significant change in body weight was observed for the test groups 11 and 12 to which the prodrug nanoparticles of Example 4 were administered, and significant body weight decrease by 9% or more was observed for the test groups 9 and 10 to which the anticancer agent was administered alone.

As seen from FIG. 18, the heart tissue of the test groups 9 and 10 to which the anticancer agent (DOX) was administered alone was damaged severely, and the kidney tissue, etc. were also damaged significantly. Specifically, the organ tissues of the test groups 9 and 10 showed substantially complete destruction of micromorphology, and the dysfunction of biological function was induced. In contrast, the test groups 11 and 12 to which the prodrug nanoparticles of Example 4 were administered showed no damaged organ tissue. That is to say, it can be seen that the prodrug nanoparticles of Example 4 are very stable for normal tissues and do not cause side effects in vivo.

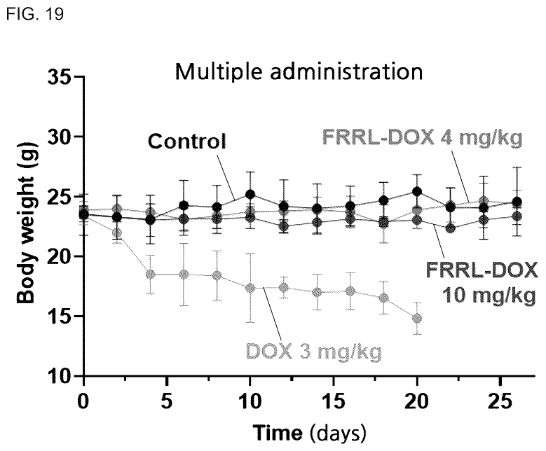

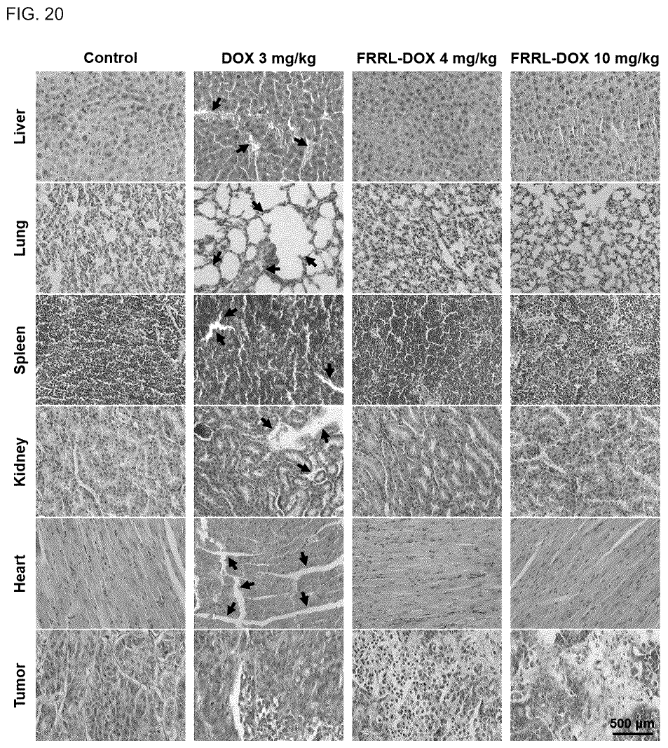

FIG. 19 shows the result of measuring body weight change for the normal group and the test groups 13 to 16. FIG. 20 shows the result of the histochemical analysis result of the liver, lung, spleen, kidney and heart tissues of the normal group and the test groups 13 to 16 stained with H&E.

As seen from FIG. 19, for the test groups 9 and 10 to which the anticancer agent (DOX) was administered alone, rapid body weight decrease was induced from day 5 and all the mice died within 20 days. It can be seen that, for the existing anticancer agent, the risk of side effects increases significantly and death is induced in the normal group in the case of multiple administration. That is, even death may be induced when the anticancer agent is administered repeatedly to achieve the efficacy for complete prevention and treatment of cancer.

In contrast, the test groups 11 and 12 to which the prodrug nanoparticles of Example 4 were administered showed no body weight change and all the mice survived healthily for more than 26 days. The test group 12 showed stability although they were treated with 10 mg/kg of prodrug nanoparticles (Example 4) (excess anticancer agent) with 3 times higher concentration of the anticancer agent.

As seen from FIG. 20, whereas normal tissues were destroyed and damaged for the test groups 9 and 10 to which the anticancer agent (DOX) was administered alone, no significant damage of organ tissues was observed for the test groups 11 and 12 to which the prodrug nanoparticles of Example 4 were administered. That is to say, it can be seen that the prodrug nanoparticles of Example 4 maintain inactive state stably in normal tissues even when they are administered repeatedly, and are specifically activated in the cancer tissues.

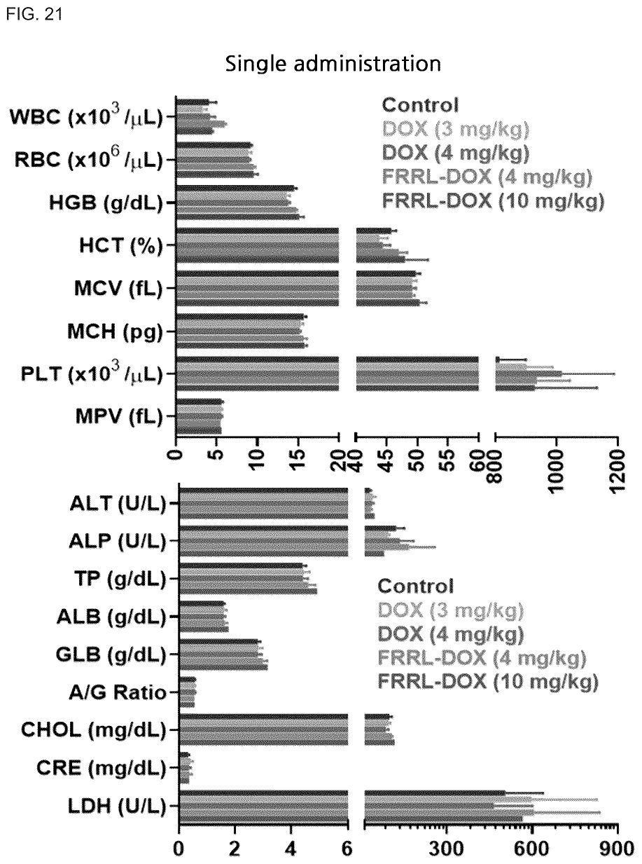

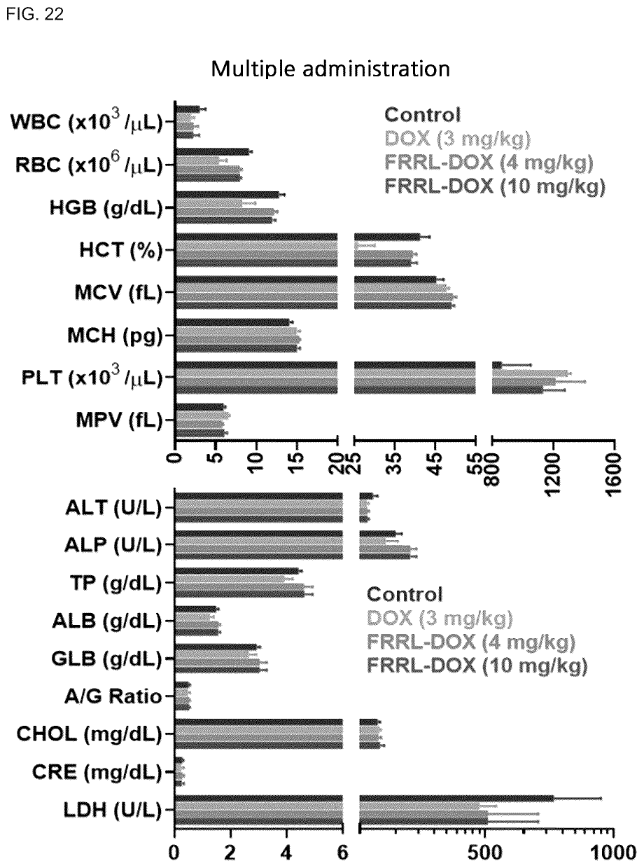

FIG. 21 shows the result of evaluating hemocompatibility for the normal group and the test groups 9 to 12. FIG. 22 shows the result of evaluating hemocompatibility for the normal group and the test groups 13 to 16.

As seen from FIG. 21, the test groups 9 to 12 showed no hematotoxicity for single administration.

As seen from FIG. 22, the test groups 13 and 14 showed significant hematotoxicity for multiple administration. Specifically, for the test groups 13 and 14 to which the anticancer agent (DOX) was administered alone, the levels of hemoglobin and hematocrit were outside normal ranges. In contrast, the test groups 15 and 16 to which the prodrug nanoparticles of Example 4 were administered showed hemocompatibility with no hematotoxicity induced regardless of the administration method.

1. A cancer-targeting peptide represented by General Formula 1 that can be cleaved by cathepsin B in cancer cells:
Phe-[Xaa1]ₙ-Arg-Arg-[Xaa2]ₘ-[Gly]ₒ [General Formula 1]
In the formula 1,
each of Xaa1 and Xaa2 is independently Leu or Gly,
each of n to o is independently 0 or 1,
with the proviso that n, m and o are not 0 at the same time, and
if n is 0, m is 1 and Xaa2 is Leu.

2. The cancer-targeting peptide according to claim 1, wherein, in the formula 1, n+m+o is 2 or smaller.

3. The cancer-targeting peptide according to claim 1, wherein, in the formula 1, if n is 1, any one of m or o is necessarily 1.

4. The cancer-targeting peptide according to claim 1, wherein the peptide is any one selected from amino acid sequences represented by SEQ ID NOS: 1 to 3.

5. The cancer-targeting peptide according to claim 1, wherein the peptide is represented by SEQ ID NO: 1.

6. A conjugate wherein a peptide represented by General Formula 1 is conjugated with an anticancer agent:
Phe-[Xaa1]ₙ-Arg-Arg-[Xaa2]ₘ-[Gly]ₒ [General Formula 1]
In the formula 1,
each of Xaa1 and Xaa2 is independently Leu or Gly,
each of n to o is independently 0 or 1,
with the proviso that n, m and o are not 0 at the same time, and
if n is 0, m is 1 and Xaa2 is Leu.

7. The conjugate according to claim 6, wherein, in the formula 1, n+m+o is 2 or smaller.

8. The conjugate according to claim 6, wherein, in the formula 1, if n is 1, any one of m or o is necessarily 1.

9. The conjugate according to claim 6, wherein the peptide is any one selected from amino acid sequences represented by SEQ ID NOS: 1 to 3.

10. The conjugate according to claim 6, wherein the peptide is represented by SEQ ID NO: 1.

11. The conjugate according to claim 6, wherein the conjugate is prepared into a prodrug nanoparticle in a solution through self-assembly.

12. The conjugate according to claim 11, wherein the prodrug nanoparticles have an average diameter of 130 to 170 nm.

13. The conjugate according to claim 11, wherein the proportion of the hydrophobic surface in the entire molecular surface area of the prodrug nanoparticles is 55 to 60%.

14. The conjugate according to claim 6, wherein the anticancer agent is any one selected from a group consisting of Taxol, bendamustine, busulfan, carmustine, chlorambucil, cyclophosphamide, dacarbazine, Adriamycin, daunomycin, ifosfamide, melphalan, procarbazine, streptozocin, temozolomide, asparaginase, capecitabine, cytarabine, 5-fluorouracil, fludarabine, gemcitabine, methotrexate, pemetrexed, raltitrexed, actinomycin D, bleomycin, daunorubicin, doxorubicin, PEGylated liposomal doxorubicin, epirubicin, idarubicin, mitomycin, mitoxantrone, etoposide, docetaxel, irinotecan, paclitaxel, topotecan, vinblastine, vincristine, vinorelbine, carboplatin, cisplatin, oxaliplatin, alemtuzumab, BCG, bevacizumab, cetuximab, denosumab, erlotinib, gefitinib, imatinib, interferon, ipilimumab, lapatinib, panitumumab, rituximab, sunitinib, sorafenib, temsirolimus, trastuzumab, clodronate, ibandronic acid, pamidronate and zoledronic acid.

15. A pharmaceutical composition for preventing or treating cancer, comprising the conjugate according to claim 6 as an active ingredient.

16. The pharmaceutical composition for preventing or treating cancer according to claim 15, wherein the conjugate is activated as it is degraded by cathepsin B present in cancer cells.

17. The pharmaceutical composition for preventing or treating cancer according to claim 15, wherein the pharmaceutical composition is accumulated 2 times or more in cancer tissue than in kidney tissue or liver tissue.

18. The pharmaceutical composition for preventing or treating cancer according to claim 15, wherein the cancer is one or more selected from a group consisting of lung cancer, stomach cancer, glioma, liver cancer, melanoma, kidney cancer, urothelial cancer, head and neck cancer, Merkel cell carcinoma, prostate cancer, blood cancer, breast cancer, mammary gland cancer, colorectal cancer, colon cancer, rectal cancer, pancreatic cancer, brain cancer, ovarian cancer, bladder cancer, bronchial cancer, skin cancer, cervical cancer, endometrial cancer, esophageal cancer, adenocarcinoma of the nasopharynx, thyroid cancer, bone cancer and combinations thereof.
